SEQUENCE LISTING (13 sequences)

SEQ ID NO: 1 - protein, 182 aa, Bacillus cibi [sequence omitted]

SEQ ID NO: 2 - protein, 182 aa, Artificial Sequence
  Synthesized/synthetic construct
IPPGTPSKSAAQYQLNALTVKPEGSMLGYSRDKFPHWIPQGGGCDTRQVVLKRDAIWYVGNCPVVSGSWYSYYDGLTFTNPSDLDIDHIVPLAEAWRSGASSWTTSKRRDFANDLDGPQLIAVSASVNRSKGDQDPSTWQPPRPGAHCGYSKWWISTKYKWGLSLQLSEKTALQDMLNSCSY

SEQ ID NO: 3 - protein, 182 aa, Artificial Sequence
  Synthesized/synthetic construct
IPPGTPSKSAAQSQLNALTVKPEGSMSGYSRDLFPHWISQGSGCDTRQVVLKRDADWYSGNCPVTSGSWYSYYDGLTFTNPSDLDIDHIVPLAEAWRSGASSWTTSKRQDFANDLSGPQLIAVSASTNRSKGDQDPSTWQPPRSGAHCGYSKWWISTKYKWGLSLQLSEKTALQDMLNSCSY

SEQ ID NO: 4 - protein, 182 aa, Artificial Sequence
  Synthesized/synthetic construct
IPPGTPSKSAAQYQLNALTVKPEGSMLGYSRDLFPHWISQGGGCDTRQVVLKRDADWYVGNCPVTSGSWYSYYDGLTFTNPSDLDIDHIVPLAEAWRSGASSWTTSKRRDFANDLDGPQLIAVSASVNRSKGDQDPSTWQPPRPGAHCGYSKWWISTKYKWGLSLQLSEKTALQDMLNSCSY

SEQ ID NO: 5 - protein, 182 aa, Artificial Sequence
  Synthesized/synthetic construct
IPPGTPSKSAAQYQLNALTVKPEGPMLGYSRDLFPHWIPQGGGCDTRQVVLKRDADWYVGNCPVVYGSWYSYYDGLYFTNPSDLDIDHIVPLAEAWRSGASSWTTSKRRDFANDLDGPQLIAVSASVNRSKGDQDPSTWQPPRPGAHCGYSKWWISTKYKWGLSLQLSEKTALQDMLNSCSY

SEQ ID NO: 6 - protein, 182 aa, Artificial Sequence
  Synthesized/synthetic construct
IPPGTPSKSAAQYQLNALTVKPEGSMLGYSRDLFPHWIPQGGGCDTRQVVLKRDALWYVGNCPVVSGSWYSYYDGLTFTNPSDLDIDHIVPLAEAWRSGASSWTTSKRRDFANDLDGPQLIAVSASVNRSKGDQDPSTWQPPRPGAHCGYSKWWISTKYKWDLSLQLSEKTALQDMLNSCSY

SEQ ID NO: 7 - protein, 182 aa, Artificial Sequence
  Synthesized/synthetic construct
IPPGTPSKSAAQYQLNALTVKPEGSMRGYSRDLFPHWIPQGGGCDTRQVVLKRDALWYVGNCPVVSGSWYSYYDGLTFTNPSDLDIDHIVPLAEAWRSGASSWTTSKRRDFANDLDGPQLIAVSASVNRSKGDQDPSTWQPPRPGAHCGYSKWWISTKYKWDLSLQLSEKTALQDMLNSCSY

SEQ ID NO: 8 - protein, 204 aa, Aspergillus oryzae
KTGSGDSQSDPIKADLEVKGQSALPFDVDCWAILCKGAPNVLQRVNEKTKNSNRDRSGANKGPFKDPQKWGIKALPPKNPSWSAQDFKSPEEYAFASSLQGGTNAILAPVNLASQNSQGGVLNGFYSANKVAQFDPSKPQQTKGTWFQITKFTGAAGPYCKALGSNDKSVCDKNKNIAGDWGFDPAKWAYQYDEKNNKFNYVGK

SEQ ID NO: 9 - protein, 221 aa, Aspergillus oryzae
VPVNPEPDATSVENVALKTGSGDSQSDPIKADLEVKGQSALPFDVDCWAILCKGAPNVLQRVNEKTKNSNRDRSGANKGPFKDPQKWGIKALPPKNPSWSAQDFKSPEEYAFASSLQGGTNAILAPVNLASQNSQGGVLNGFYSANKVAQFDPSKPQQTKGTWFQITKFTGAAGPYCKALGSNDKSVCDKNKNIAGDWGFDPAKWAYQYDEKNNKFNYVGK

SEQ ID NO: 10 - protein, 188 aa, Trichoderma harzianum
APAPMPTPPGIPTESSARTQLAGLTVAVAGSGTGYSRDLFPTWDAISGNCNAREYVLKRDGEGVQVNNACESQSGTWISPYDNASFTNASSLDIDHMVPLKNAWISGASSWTTAQREALANDVSRPQLWAVSASANRSKGDRSPDQWKPPLTSFYCTYAKSWIDVKSFYKLTITSAEKTALSSMLDTC

SEQ ID NO: 11 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 12 - protein, 275 aa, Bacillus amyloliquefaciens [sequence omitted]

SEQ ID NO: 13 - protein, 298 aa, Bacillus bogoriensis
ANSGFYVSGTTLYDANGNPFVMRGINHGHAWYKDQATTAIEGIANTGANTVRIVLSDGGQWTKDDIHTVRNLISLAEDNHLVAVLEVHDATGYDSIASLNRAVDYWIEMRSALIGKEDTVIINIANEWFGSWEGDAWADGYKQAIPRLRNAGLNHTLMVDAAGWGQFPQSIHDYGREVFNADPQRNTMFSIHMYEYAGGNASQVRTNIDRVLNQDLALVIGEFGHRHTNGDVDEATIMSYSEQRGVGWLAWSWKGNGPEWEYLDLSNDWAGNNLTAWGNTIVNGPYGLRETSRLSTVF

(19) United States
(12) Patent Application Publication
(10) Pub. No.: US 2023/0357674 A1
(43) Pub. Date: Nov. 9, 2023

(54) MEDICAL CLEANING COMPOSITION, USE AND METHOD OF CLEANING

(71) Applicant: Novozymes A/S, Bagsvaerd (DK)

(72) Inventors: Rasmus Rune Hansen, Ballerup (DK); Inge Byg Ulbrink, Bagsvaerd (DK)

(73) Assignee: Novozymes A/S, Bagsvaerd (DK)

(21) Appl. No.: 17/923,489

(22) PCT Filed: May 6, 2021
(86) PCT No.: PCT/EP2021/061988

§ 371 Date: Nov. 4, 2022

(30) Foreign Application Priority Data
May 7, 2020 (EP) 20173450.6

(51) Int. Cl. C11D 3/48; C11D 3/386; C11D 11/00; C12N 9/22; C11D 3/39

(52) U.S. Cl. CPC C11D 3/38636; C11D 3/3945; C11D 3/3947; C11D 3/48; C11D 11/0023; C12N 9/22; C12Y 301/21001

(57) ABSTRACT

The invention relates to methods for cleaning a medical device, a medical cleaning composition, use of a polypeptide having DNase activity for cleaning a medical device, and a medical device coated with a polypeptide having DNase activity.

### FIELD OF THE INVENTION

The present invention relates to a method for cleaning a medical device, a medical cleaning composition, use of a polypeptide having DNase activity for cleaning a medical device, and a medical device coated with a polypeptide having DNase activity.

### BACKGROUND

Medical devices are often heavily contaminated with organic soil as a result of their use. Before re-use, it is essential that the devices are properly cleaned and disinfected.

The nature and extent of contamination in the healthcare environment is far greater than that normally found in a domestic environment, and the need for efficient cleaning is high. Of particular concern are microorganisms which are resistant to multiple types of antibiotics as these are over-represented in healthcare environments. Accordingly, use of unsufficiently reprocessed medical equipment poses a high risk of cross-infection to other patients, whose immune systems may already be compromised by illness, injury or the trauma of invasive medical procedures.

Typically, the procedure for reprocessing of medical devices comprises washing the device to remove organic materials, rinsing, disinfection and drying. Cleaned medical devices are often still soiled with organic material, which can significantly decrease the efficacy of the subsequent disinfection procedure.

WO2017/129331 relates to cleaning of medical and dental instruments with protease.

WO 2014/110015 relates to compositions comprising a protease and methods for cleaning medical and dental instruments.

### SUMMARY

The present invention pertains to a method for cleaning a medical device, comprising the steps of:
a) providing a wash liquor having a pH in the range of 6.0-10.0, wherein the wash liquor comprises a polypeptide having DNase activity and optionally further cleaning components,
b) subjecting at least part of the medical device to the wash liquor of step a),
c) optionally, subjecting the medical device to rinsing,
d) optionally, subjecting the medical device to drying, and
e) optionally, subjecting the medical device to sterile packaging.

In one aspect the invention relates to a medical cleaning composition comprising a polypeptide having DNase activity, wherein the composition has a pH in the range of 6.0-10.0.

In a further aspect the invention concerns use of a polypeptide having DNase activity for the cleaning at least part of a medical device.

In another aspect, the invention pertains to a medical device coated with a composition comprising a polypeptide having DNase activity.

### OVERVIEW OF FIGURES

FIG. 1: Percent remaining dead biofilm after 60 minutes treatment with 0.5% model detergent with and without enzymes.

FIG. 2: Percent remaining dead biofilm from P. aeruginosa PA14 (grown for 4 hours or 24 hours before kill) after 60 minutes treatment with 0.5% model detergent ± DNase SEQ ID NO: 1 in dosages from 0 to 5 µg/mL. Left hand columns: 4 hours, right hand columns: 24 hours.

FIG. 3: Percent remaining dead biofilm from P. aeruginosa PA01 (grown for 4 hours or 24 hours before kill) after 60 minutes treatment with 0.5% model detergent ± DNase SEQ ID NO: 1 in dosages from 0 to 5 µg/mL. Left hand columns: 4 hours, right hand columns: 24 hours.

FIG. 4: Percent remaining dead biofilm from P. aeruginosa PA14 (grown for 4 hours or 24 hours before kill) after 60 minutes treatment with 0.5% model detergent ± DNase SEQ ID NO: 2 in dosages from 0 to 5 µg/mL. Left hand columns: 4 hours, right hand columns: 24 hours.

FIG. 5: Percent remaining dead biofilm from P. aeruginosa PA01 (grown for 4 hours or 24 hours before kill) after 60 minutes treatment with 0.5% model detergent ± DNase SEQ ID NO: 2 in dosages from 0 to 5 µg/mL. Left hand columns: 4 hours, right hand columns: 24 hours.

FIG. 6: Percent remaining dead biofilm from P. aeruginosa PA14 (grown for 4 hours or 24 hours kill) after 60 minutes treatment with 0.5% model detergent ± DNase SEQ ID NO: 9 in dosages from 0 to 5 µg/mL. Left hand columns: 4 hours, right hand columns: 24 hours.

FIG. 7: Percent remaining dead biofilm from P. aeruginosa PA01 (grown for 4 hours or 24 hours before kill) after 60 minutes treatment with 0.5% model detergent ± DNase SEQ ID NO: 9 in dosages from 0 to 5 µg/mL. Left hand columns: 4 hours, right hand columns: 24 hours.

FIG. 8: Absorbance of crystal violet stained dead biofilm from P. aeruginosa PA14 (grown for 4 hours before kill) after treatment with DNase SEQ ID NO: 2 in dosages from 0 - 5 ppm in endoscope reprocessing EU detergent 1, Dr. Weigert Neodisher Multizym.

FIG. 9: Absorbance of crystal violet stained dead biofilm from P. aeruginosa PA14 (grown for 4 hours before kill) after treatment with DNase SEQ ID NO: 2 in dosages from 0 - 5 ppm in endoscope reprocessing EU detergent 2, Aniosyme Synergy 5.

FIG. 10: Absorbance of crystal violet stained dead biofilm from P. aeruginosa PA14 (grown for 4 hours before kill) after treatment with DNase SEQ ID NO: 2 in dosages from 0 - 5 ppm in endoscope reprocessing US detergent 1, Steris Prolystica 2X Enzymatic Presoak and Cleaner.

FIG. 11: Absorbance of crystal violet stained dead biofilm from P. aeruginosa PA14 (grown for 4 hours before kill) after treatment with DNase SEQ ID NO: 2 in dosages from 0 - 5 ppm in endoscope reprocessing US detergent 2, Metrex Metrizyme.

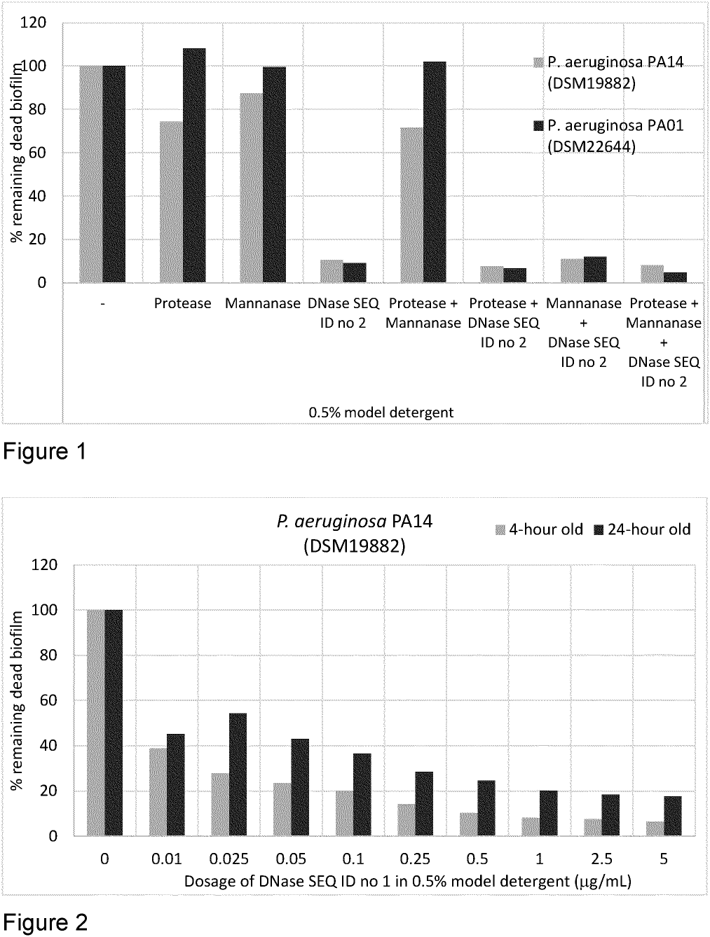

### DEFINITIONS

“At least the part of the medical device”: This term is to be understood as the part of a medical device being in contact with the patient, or the interior of e.g. an endoscope which is in contact with fluids or samples which have been in contact with the patient.

Medical cleaning composition: The term “cleaning composition” includes “detergent composition” and refers to compositions that find use in the removal of undesired compounds from items to be cleaned, such as medical devices. The terms encompass any materials/compounds selected for the particular type of cleaning composition desired and the form of the product (e.g., liquid, gel, powder, granulate, paste, or spray compositions). The medical cleaning composition may comprise one or more cleaning components.

Medical cleaning component: The medical cleaning component is a component different to the DNase enzyme. The precise nature of medical cleaning components, and levels of incorporation thereof, will depend on the physical form of the composition and the nature of the operation for which it is to be used. Suitable cleaning components include, but are not limited to, the components described below under there respective headers. These includes wetting agents also known as surfactants, sequesting agents known as chelators or builders, which are used for reducing water hardness, dispersants, polymeric agents, carriers, hydrotropes, processing aids, bactericides, fungicides, soil suspending agents, anti-corrosion agents, bluing agents and fluorescent dyes, antioxidants, and solubilizers.

In addition to containing the polypeptide having DNase, protease or mannanase activity, the medical cleaning composition may contain one or more additional enzymes, such as amylases, lipases, cutinases, cellulases, endoglucanases, xyloglucanases, pectinases, pectin lyases, xanthanases, peroxidases, haloperoxygenases, catalases, galactanase or any mixture thereof), enzyme stabilizers and enzyme inhibitors, in particular protease inhibitors.

Mature polypeptide: The term “mature polypeptide” means a polypeptide in its final form following translation and any post-translational modifications, such as N-terminal processing, C-terminal truncation, glycosylation, phosphorylation, etc.

Nomenclature: For purposes of the present invention, the nomenclature [E/Q] or [EQ] means that the amino acid at this position may be a glutamic acid (Glu, E) or a glutamine (Gln, Q). Likewise, the nomenclature [V/G/A/I] or [VGAI] means that the amino acid at this position may be a valine (Val, V), glycine (Gly, G), alanine (Ala, A) or isoleucine (Ile, I), and so forth for other combinations as described herein. Further, the amino acid X is defined such that it may be any of the 20 natural amino acids.

Polypeptide having DNase activity: Polypeptides having DNase activity, also referred to as DNases, are enzymes that catalyze the hydrolytic cleavage of phosphodiester linkages in the DNA backbone, thus degrading DNA. The terms “DNase” and “polypeptide having DNase activity” can be used interchangeably. For purposes of the present invention, DNase activity is determined according to the procedure described in the Assay I.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows: (Identical Residues x 100)/(Length of Alignment - Total Number of Gaps in Alignment)

Variant: The term “variant” means a polypeptide having enzyme activity comprising an alteration, i.e., a substitution, insertion, and/or deletion, at one or more positions. A substitution means replacement of the amino acid occupying a position with a different amino acid; a deletion means removal of the amino acid occupying a position; and an insertion means adding an amino acid adjacent to and immediately following the amino acid occupying a position.

Wash liquor: The term “wash liquor” is defined herein as a solution or mixture of water and a cleaning composition.

### DETAILED DESCRIPTION

In one aspect, the present invention relates to a method for cleaning a medical device comprising the steps of:
a) providing a wash liquor having a pH in the range of 6.0-10.0, wherein the wash liquor comprises a polypeptide having DNase activity and optionally further cleaning components,
b) subjecting at least part of the medical device to the wash liquor of step a),
c) optionally, subjecting the medical device to rinsing,
d) optionally, subjecting the medical device to drying, and
e) optionally, subjecting the medical device to sterile packaging.

Medical devices can be heavily soiled with organic soil. The organic soil may comprise material of organic origin such as biofilm or material from a patient. The patient can be an animal or human being. The material may be of human or animal origin, e.g. combinations of organic material from an animal or a human, such as blood, mucus, tissue, adipose tissue, urine, faeces and microorganisms e.g. bacteria. The organic material may adhere to the part of the medical device that has been into contact with the patient, or to the interior of e.g. an endoscope which is in contact with fluids or samples from the patient. The organic material may serve as nutrients for microorganisms present on the medical device or supplied from the use of the medical device on a patient.

Microorganisms may produce a “biofilm”, meaning a film produced by any group of microorganisms, in which the microbial cells stick to each other and/or stick to a surface of the medical device. These adherent microorganisms are frequently embedded within a self-produced matrix of extracellular polymeric substances (EPS). Biofilm EPS is a polymeric conglomeration generally composed of extracellular DNA, proteins, and polysaccharides. Biofilms may form on living or non-living surfaces such as the surface of a medical device. The microbial cells growing in a biofilm are physiologically distinct from planktonic cells of the same organism, which, by contrast, are single cells that may float or swim in a liquid medium. Microorganisms living in a biofilm usually have significantly different properties from planktonic bacteria of the same species, as the dense and protected environment of the film allows them to cooperate and interact in various ways. One benefit of this environment, for the microorganisms, is increased resistance to detergents and antibiotics, as the dense extracellular matrix and the outer layer of cells protect the interior of the community.

The organic soil may comprise one or more microorganisms, such as a group of microorganisms selected from the group consisting of, but not limited to Acinetobacter sp., Aeromicrobium sp., Aspergillus versicolor, Brevundimonas sp., Enterobacter cloacae, Enterococcus faecalis, Escherichia coli, Klebsiella pneumoniae, Microbacterium sp., Micrococcus luteus, Pseudomonas sp., Staphylococcus aureus, Staphylococcus epidermidis, Stenotrophomonas sp., Bacillus sp. and Pseudomonas aeruginosa. Especially Pseudomonas aeruginosa may be comprised in the organic soil.

In a preferred embodiment, the wash liquor comprises a polypeptide having DNase activity, and the polypeptide releases and/or removes at least part of the organic soil present on the part of the medical device subjected to the wash liquor.

Medical devices are typically disinfected by subjecting at least the part of the medical device soiled with organic soil to chemical, physical or biological disinfection, e.g. to autoclaving, radiation or chemical disinfectants. However, if the cleaning step has been insufficient, the traditional methods for disinfection may leave organic soil on the surface of the device. The organic soil may comprise components from the patient, such as tissue or mucus, and living or dead microorganisms.

In a preferred embodiment, the medical device is disinfected before being subjected to the wash liquor comprising the polypeptide having DNase activity. This situation occurs when the device has been used, unsufficiently cleaned, disinfected, used again and then is supposed to be cleaned. When an unsufficiently cleaned device is disinfected the remaining soil on said device will harbor an organic soil as described above. The disinfection may remove some or all of the organic soil or may leave organic soil on surfaces of the device. The disinfection may kill the microorganisms present in the organic soil, e.g. by killing microorganisms present in biofilm. Biofilm in which all microorganisms are killed is termed dead biofilm. This dead biofilm will be subject to cleaning after the next use of the device.

In a preferred embodiment, a medical device soiled with a dead biofilm will be subject to cleaning in accordance with the method of the invention.

In a preferred embodiment, the disinfection is performed by subjecting at least part of the medical device to a chemical or a biological disinfection composition or to radiation. The disinfection may be performed by subjecting at least part of the medical device to antimicrobial agents, antibiotics, fungicides, oxidizing agents, hydrogen peroxide, chlorine, acids, peroxy acids, peracetic acid, potassium permanganate, ultra-violet radiation (UV) and/or ozon.

In a preferred embodiment, the disinfection leaves substantial no living microorganisms left in the organic soil present on the medical device.

In a preferred embodiment, the medical device is subjected to a liquid solution comprising peracetic acid having a pH of about 3, such as about 3.0, whereafter no live microorganisms are comprised in the organic soil. The complete killing of microorganisms in organic soil and verification of the killing is demonstrated in example 1.

In a preferred embodiment, at least 80% of the microorganisms comprised in the organic soil are dead. In a more preferred embodiment, at least 85% of the microorganisms comprised in the organic soil are dead. In an even more preferred embodiment, at least 90% of the microorganisms comprised in the organic soil are dead. In a most preferred embodiment, at least 95% of the microorganisms comprised in the organic soil are dead. In a preferred embodiment 100% of the microorganisms in the organic soil are dead. Example 1 demonstrates how 100% of the microorganism present in the organic soil is dead.

In a preferred embodiment, the pH of the wash liquor may be in the range of 6.5-9.5. In a more preferred embodiment, the pH of the wash liquor is in the range of 7.0-9.0, even more preferably in the range of 7.5-8.5, such as a pH around 8.

The wash liquor can be provided by mixing cleaning components with water and the polypeptide having DNase activity. In a preferred embodiment, the wash liquor is provided by mixing the medical cleaning composition of the invention with a suitable amount of liquid, e.g. water.

In an embodiment of the invention, the wash liquor comprises at least 0.001 ppm of the polypeptide having DNase activity. In a preferred embodiment, the wash liquor comprises at least 0.005 ppm of the polypeptide having DNase activity, more preferably at least 0.01 ppm of the polypeptide having DNase activity, even more preferably at least 0.05 ppm of the polypeptide having DNase activity or most preferably at least 1 ppm of the polypeptide having DNase activity.

In an embodiment of the invention, the wash liquor comprises at most 100 ppm of the polypeptide having DNase activity. In a preferred embodiment, the wash liquor comprises at most 50 ppm of the polypeptide having DNase activity, more preferably at most 30 ppm of the polypeptide having DNase activity, even more preferably at most 20 of the polypeptide having DNase activity or most preferably at most 10 ppm of the polypeptide having DNase activity.

In an embodiment of the invention, the wash liquor comprises in the range of 0.001-100 ppm of the polypeptide having DNase activity. In a preferred embodiment, the wash liquor comprises in the range of 0.005-50 ppm of the polypeptide having DNase activity, more preferably in the range of 0.01-30 ppm of the polypeptide having DNase activity, even more preferably in the range of 0.05-20 ppm of the polypeptide having DNase activity or most preferably in the range of 1-10 ppm more of the polypeptide having DNase activity.

In an embodiment of the invention, the wash liquor comprises in the range of 0.05-5 ppm of the polypeptide having DNase activity. In a preferred embodiment, the wash liquor comprises in the range of 0.025-5 ppm of the polypeptide having DNase activity, more preferably in the range of 0.05-5 ppm of the polypeptide having DNase activity, even more preferably in the range of 1.0-5 ppm of the polypeptide having DNase activity or most preferably in the range of 2.5-5 ppm more of the polypeptide having DNase activity.

The wash liquor may further comprise one or more cleaning composition components, preferably selected from surfactants, builders, polymers, dispersing agents and additional enzymes.

The additional enzymes may be selected from the group consisting of proteases, amylases, lipases, cutinases, cellulases, endoglucanases, xyloglucanases, pectinases, pectin lyases, xanthanases, peroxidases, haloperoxygenases, catalases, galactanase or any mixture thereof.

In an embodiment of the invention, the wash liquor comprises in the range of 0.001-100 ppm of a polypeptide having mannanase activity. In a preferred embodiment, the wash liquor comprises in the range of 0.005-50 ppm of the polypeptide, more preferably in the range of 0.01-30 ppm of the polypeptide, even more preferably in the range of 0.05-20 ppm of the polypeptide or most preferably in the range of 1-10 ppm more of the polypeptide.

In an embodiment of the invention, the wash liquor comprises in the range of 0.001-100 ppm of a polypeptide having protease activity (also referred to herein as a protease). In a preferred embodiment, the wash liquor comprises in the range of 0.005-50 ppm of the polypeptide, more preferably in the range of 0.01-30 ppm of the polypeptide, even more preferably in the range of 0.05-20 ppm of the polypeptide or most preferably in the range of 1-10 ppm more of the polypeptide.

In one embodiment of the invention, the entire medical device is subjected to the wash liquor comprising the polypeptide having DNase activity. The medical device may comprise parts that do not tolerate to be subjected to a liquid, e.g. water or wash liquor. Thus, in some embodiments, any parts of the medical device that do not tolerate liquid are not subjected to the wash liquor comprising the polypeptide having DNase activity. In some embodiments, at least a part of the medical device is subjected to the wash liquor comprising the polypeptide having DNase activity, e.g. the part of the medical device that is in contact with the patient.

The medical device may be subjected to the wash liquor by spraying, dipping or immersing, e.g. by immersing the whole part or at least part of the medical device into the wash liquor.

The whole or at least part of the medical device is soaked in the wash liquor for a time sufficient to reduce and/or remove soil on the instrument, preferably for at least 1 minute.

The medical device is soaked in the wash liquor for a time sufficient to be effective at reducing or removing the organic soil on the device. Depending on the level of soiling, the device may be soaked in the wash liquor for at least 1 minute. Soaking may continue even after cleaning is complete. For example, for convenience cleaning may continue over night, or even for up to 24 hours. The upper time limit is determined by the robustness of the device, to avoid damaging it.

The medical device may be soaked in the wash liquor at a temperature between room temperature and 90° C., preferably between 20 and 90° C., more preferably between 30 and 80° C., even more preferably between 30 and 70° C., and most preferably between 30 and 60° C.

Soaking of the medical device may be carried out with or without mechanical action (such as shaking or stirring) in a tray, tub, pan, or sink; or by spraying, such as through an instrument washer; by ultrasonic treatment, treatment in a cart or cage washer; by manually applying it with a hand-held bottle as either a spray or a foam; or by mechanized washing in a laboratory glass machine washer.

In an embodiment, the cleaning of medical devices, and/or non-medical types of equipment, takes place in a (Medical) Washer-Disinfector according to EN ISO 15883-1 (or as described in “Class II Special Controls Guidance Document: Medical Washers and Medical Washer-Disinfectors; Guidance for the Medical Device Industry and FDA Review Staff”, U.S. Food and Drug Administration, February 2002), using the methods of the invention.

During soaking of the soiled medical device, at least part of the medical device is contacted with an effective amount of the wash liquor comprising the polypeptide having DNase activity. The actual amount of the medical cleaning composition used will be based on the judgment of the user and will depend upon factors such as the particular product formulation of the composition, the concentration of the composition, the number of soiled medical devices to be cleaned, and the degree of soiling of the articles. Subsequently, the items may be subjected to a manual or machine washing or rinsing method, involving either further washing steps and use of a cleaning composition, and/or to a manual or machine rinsing method. The disclosed compositions and methods are effective at removing the organic soil on devices in combination with mechanical action, such as e.g. scrubbing and may improve the cleaning process by reducing the amount of scrubbing required, and the overall result by a greater cleanliness of the medical device.

The wash liquor comprising the peptide having DNase activity can be employed in a variety of machines that clean medical devices. Such machines can be charged manually with liquid forms, or powder or other solid forms of the medical cleaning composition. Such machines can also automatically dispense the disclosed compositions. Such dispensing can include dissolving the medical cleaning composition to form a wash liquor, optionally diluting the wash liquor to yield a wash liquor (that is less concentrated) and diluting the second wash liquor into the wash or soak chamber to form the used wash liquor. The wash liquor can be used to clean the devices. Such dispensing can also include dissolving a solid cleaning composition once to form a wash liquor.

The medical device can be subjected to drying after being cleaned with the wash liquor comprising the peptide having DNase activity and/or being rinsed. In some embodiments, the medical device is packaged in a sterile package after being cleaned.

In an embodiment of the invention, the wash liquor comprising a polypeptide having DNase activity releases and/or removes at least 50% of the organic soil present on the part of the medical device subjected to the wash liquor. The released and/or removed organic soil can be measured as demonstrated in Example 2. In a preferred embodiment, at least 60% of the organic soil is released and/or removed, more preferably at least 70% of the organic soil is released and/or removed, even more preferably at least 80% of the organic soil is released and/or removed, most preferably at least 90% of the organic soil is released and/or removed, such as at least 95% of the organic soil is released and/or removed.

In an embodiment of the invention, the wash liquor comprising a polypeptide having DNase activity and an additional enzyme releases and/or removes at least 50% of the organic soil present (e.g. dead biofilm) on the part of the medical device subjected to the wash liquor. The released and/or removed organic soil can be measured as demonstrated in Example 1. In a preferred embodiment, preferably at least 60% of the organic soil is released and/or removed, more preferably at least 70% of the organic soil is released and/or removed, even more preferably at least 80% of the organic soil is released and/or removed, most preferably at least 90% of the organic soil is released and/or removed, such as at least 95% of the organic soil is released and/or removed.

In a preferred embodiment, the invention relates to a method for cleaning a medical device comprising the steps of:
a) Providing a wash liquor having a pH in the range of 6.0-10.0, comprising a polypeptide having DNase activity and optionally further cleaning components,
b) Subjecting at least part of the medical device to the wash liquor of step a),
c) Optionally, subjecting the medical device to rinsing,
d) Optionally, subjecting the medical device to drying,
e) Optionally, subjecting the medical device to sterile packaging, and
wherein the wash liquor releases and/or removes at least 50% of the organic soil present on the part of the medical device subjected to the wash liquor. In a more preferred embodiment at least 60% of the organic soil is released and/or removed, more preferably at least 70% of the organic soil is released and/or removed, even more preferably at least 80% of the organic soil is released and/or removed, most preferably at least 90% of the organic soil is released and/or removed, such as at least 95% of the organic soil is released and/or removed.

In another preferred embodiment, the invention relates to a method for cleaning a medical device comprising the steps of:
a) Providing a wash liquor having a pH in the range of 6.0-10.0, comprising a polypeptide having DNase activity, a polypeptide having protease activity and/or a polypeptide having mannanase activity and optionally further cleaning components,
b) Subjecting at least part of the medical device to the wash liquor of step a),
c) Optionally, subjecting the medical device to rinsing,
d) Optionally, subjecting the medical device to drying,
e) Optionally, subjecting the medical device to sterile packaging, and
wherein the wash liquor releases and/or removes at least 50% of the organic soil present on the part of the medical device subjected to the wash liquor. In a more preferred embodiment at least 60% of the organic soil is released and/or removed, more preferably at least 70% of the organic soil is released and/or removed, even more preferably at least 80% of the organic soil is released and/or removed, most preferably at least 90% of the organic soil is released and/or removed, such as at least 95% of the organic soil is released and/or removed.

In another preferred embodiment, the invention relates to a method for cleaning a medical device comprising the steps of:
a) Providing a wash liquor having a pH in the range of 6.0-10.0, comprising a polypeptide having DNase activity and a polypeptide having protease activity and/or a polypeptide having mannanase activity and optionally further cleaning components,
b) Subjecting at least part of the medical device to the wash liquor of step a),
c) Optionally, subjecting the medical device to rinsing,
d) Optionally, subjecting the medical device to drying,
e) Optionally, subjecting the medical device to sterile packaging, and
wherein the wash liquor releases and/or removes at least 50% of the organic soil present on the part of the medical device subjected to the wash liquor. In a more preferred embodiment at least 60% of the organic soil is released and/or removed, more preferably at least 70% of the organic soil is released and/or removed, even more preferably at least 80% of the organic soil is released and/or removed, most preferably at least 90% of the organic soil is released and/or removed, such as at least 95% of the organic soil is released and/or removed.

In a preferred embodiment, the invention relates to a method for cleaning a medical device comprising the steps of:
a) Providing a wash liquor having a pH in the range of 6.0-10.0, comprising a polypeptide having DNase activity and optionally further cleaning components,
b) Subjecting at least part of the medical device to the wash liquor of step a),
c) Subjecting the medical device to rinsing,
d) Optionally, subjecting the medical device to drying,
e) Optionally, subjecting the medical device to sterile packaging, and
wherein the wash liquor releases and/or removes at least 50% of the organic soil present on the part of the medical device subjected to the wash liquor. In a more preferred embodiment at least 60% of the organic soil is released and/or removed, more preferably at least 70% of the organic soil is released and/or removed, even more preferably at least 80% of the organic soil is released and/or removed, most preferably at least 90% of the organic soil is released and/or removed, such as at least 95% of the organic soil is released and/or removed.

In another preferred embodiment, the invention relates to a method for cleaning a medical device comprising the steps of:
a) Providing a wash liquor having a pH in the range of 6.0-10.0, comprising a polypeptide having DNase activity, a polypeptide of having protease activity and/or a polypeptide having mannanase activity and optionally further cleaning components,
b) Subjecting at least part of the medical device to the wash liquor of step a),
c) Subjecting the medical device to rinsing,
d) Optionally, subjecting the medical device to drying,
e) Optionally, subjecting the medical device to sterile packaging, and
wherein the wash liquor releases and/or removes at least 50% of the organic soil present on the part of the medical device subjected to the wash liquor. In a more preferred embodiment at least 60% of the organic soil is released and/or removed, more preferably at least 70% of the organic soil is released and/or removed, even more preferably at least 80% of the organic soil is released and/or removed, most preferably at least 90% of the organic soil is released and/or removed, such as at least 95% of the organic soil is released and/or removed.

The medical devices that may be cleaned according to the invention include devices, instruments or equipment, including any of the various medical or surgical instruments or devices that can benefit from cleaning with the wash liquor comprising the polypeptide having DNase activity. Exemplary medical devices include instruments, devices, tools, appliances, apparatus and equipment used in medicine or surgery, including those than can be cold sterilized, soaked or washed and then heat sterilized, or otherwise benefit from cleaning in the disclosed compositions. These various instruments, devices and equipment include, but are not limited to: diagnostic instruments, trays, pans, holders, racks, forceps, scissors, shears, saws (e.g. bone saws and their blades), hemostats, knives, chisels, rongeurs, files, nippers, drills, drill bits, rasps, burrs, spreaders, breakers, elevators, clamps, needle holders, carriers, clips, hooks, gouges, curettes, retractors, straightener, punches, extractors, scoops, keratomes, spatulas, expressors, trocars, dilators, cages, glassware, tubing, catheters, cannulas, plugs, stents, endoscopes, arthoscopes and related equipment, and the like, or combinations thereof.

The medical device may be an indwelling device. The medical device can be selected from the group consisting of a catheter such as a central venous catheter, intravascular catheter, urinary catheter, Hickman catheter, peritoneal dialysis catheter, endrotracheal catheter, or wherein the device is a mechanical heart valve, a cardiac pacemaker, an arteriovenous shunt, a scleral buckle, a prosthetic joint, a tympanostomy tube, a tracheostomy tube, a voice prosthetic, a penile prosthetic, an artificial urinary sphincter, a synthetic pubovaginal sling, a surgical suture, a bone anchor, a bone screw, an intraocular lens, a contact lens, an intrauterine device, an aortofemoral graft, a vascular graft, a needle, a Luer-Lok connector, a needleless connector, surgical instrument, diagnostic instruments, trays, pans, holders, racks, forceps, scissors, shears, saws (e.g. bone saws and their blades), hemostats, knives, chisels, rongeurs, files, nippers, drills, drill bits, rasps, burrs, spreaders, breakers, elevators, clamps, needle holders, carriers, clips, hooks, gouges, curettes, retractors, straightener, punches, extractors, scoops, keratomes, spatulas, expressors, trocars, dilators, cages, glassware, tubing, catheters, cannulas, plugs, stents, endoscopes, arthoscopes and related equipment.

In one embodiment, the invention relates to a method for cleaning a medical device comprising the steps of:
a) Providing a wash liquor having a pH in the range of 6.0-10.0, comprising a polypeptide having DNase activity and optionally further cleaning components,
b) Subjecting at least part of the medical device to the wash liquor of step a),
c) Optionally, subjecting the medical device to rinsing,
d) Optionally, subjecting the medical device to drying, and
e) Optionally, subjecting the medical device to sterile packaging,
wherein the polypeptide having DNase activity is of bacterial or fungal origin.

In one embodiment, the invention relates to a method for cleaning a medical device comprising the steps of:
a) Providing a wash liquor having a pH in the range of 6.0-10.0, comprising a polypeptide having DNase activity, a polypeptide having protease activity and/or a polypeptide having mannanase activity and optionally further cleaning components,
b) Subjecting at least part of the medical device to the wash liquor of step a),
c) Optionally, subjecting the medical device to rinsing,
d) Optionally, subjecting the medical device to drying, and
e) Optionally, subjecting the medical device to sterile packaging,
wherein the polypeptide having DNase activity is of bacterial or fungal origin.

In one embodiment, the invention relates to a method for cleaning a medical device comprising the steps of:
a) Providing a wash liquor having a pH in the range of 6.0-10.0, comprising a polypeptide having DNase activity and/or a polypeptide having protease activity and optionally further cleaning components,
b) Subjecting at least part of the medical device to the wash liquor of step a),
c) Optionally, subjecting the medical device to rinsing,
d) Optionally, subjecting the medical device to drying, and
e) Optionally, subjecting the medical device to sterile packaging,
wherein the polypeptide having DNase activity is of bacterial or fungal origin.

In one embodiment, the invention relates to a method for cleaning a medical device comprising the steps of:
a) Providing a wash liquor having a pH in the range of 6.0-10.0, comprising a polypeptide having DNase activity, and/or a polypeptide having mannanase activity and optionally further cleaning components,
b) Subjecting at least part of the medical device to the wash liquor of step a),
c) Optionally, subjecting the medical device to rinsing,
d) Optionally, subjecting the medical device to drying, and
e) Optionally, subjecting the medical device to sterile packaging,
wherein the polypeptide having DNase activity is of bacterial or fungal origin.

In a preferred embodiment, the invention relates to a method for cleaning a medical device comprising the steps of:
a) Providing a wash liquor having a pH in the range of 6.0-10.0, comprising a polypeptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to any of the polypeptides shown in SEQ ID NOs: 1, 2, 3, 4, 5, 6, 7, 8, 9 or 10, wherein the polypeptide has DNase activity, and optionally comprising further medical cleaning components,
b) Subjecting at least part of the medical device to the wash liquor of step a),
c) Optionally, subjecting the medical device to rinsing,
d) Optionally, subjecting the medical device to drying,
e) Optionally, subjecting the medical device to sterile packaging, and
wherein the wash liquor releases and/or removes at least 50% of the organic soil present on the part of the medical device subjected to the wash liquor. In a more preferred embodiment at least 60% of the organic soil is released and/or removed, more preferably at least 70% of the organic soil is released and/or removed, even more preferably at least 80% of the organic soil is released and/or removed, most preferably at least 90% of the organic soil is released and/or removed, such as at least 95% of the organic soil is released and/or removed.

In a preferred embodiment, the invention relates to a method for cleaning a medical device comprising the steps of:
a) Providing a wash liquor having a pH in the range of 6.0-10.0, comprising a polypeptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the polypeptide shown in SEQ ID NO: 1, wherein the polypeptide has DNase activity, and optionally comprising further medical cleaning components,
b) Subjecting at least part of the medical device to the wash liquor of step a),
c) Optionally, subjecting the medical device to rinsing,
d) Optionally, subjecting the medical device to drying,
e) Optionally, subjecting the medical device to sterile packaging, and
wherein the wash liquor releases and/or removes at least 50% of the organic soil present on the part of the medical device subjected to the wash liquor. In a more preferred embodiment at least 60% of the organic soil is released and/or removed, more preferably at least 70% of the organic soil is released and/or removed, even more preferably at least 80% of the organic soil is released and/or removed, most preferably at least 90% of the organic soil is released and/or removed, such as at least 95% of the organic soil is released and/or removed.

In a preferred embodiment, the invention relates to a method for cleaning a medical device comprising the steps of:
a) Providing a wash liquor having a pH in the range of 6.0-10.0, comprising a polypeptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the polypeptide shown in SEQ ID NO: 8, wherein the polypeptide has DNase activity, and optionally comprising further medical cleaning components,
b) Subjecting at least part of the medical device to the wash liquor of step a),
c) Optionally, subjecting the medical device to rinsing,
d) Optionally, subjecting the medical device to drying,
e) Optionally, subjecting the medical device to sterile packaging, and
wherein the wash liquor releases and/or removes at least 50% of the organic soil present on the part of the medical device subjected to the wash liquor. In a more preferred embodiment at least 60% of the organic soil is released and/or removed, more preferably at least 70% of the organic soil is released and/or removed, even more preferably at least 80% of the organic soil is released and/or removed, most preferably at least 90% of the organic soil is released and/or removed, such as at least 95% of the organic soil is released and/or removed.

In another preferred embodiment, the invention relates to a method for cleaning a medical device comprising the steps of:
a) Providing a wash liquor having a pH in the range of 6.0-10.0, comprising a polypeptide having a sequence identity to the at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to any of the polypeptides shown in SEQ ID NOs: 1, 2, 3, 4, 5, 6, 7, 8, 9 or 10, wherein the polypeptide has DNase activity, and comprising a further polypeptide having a sequence identity to the polypeptide shown in SEQ ID NO: 11 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, wherein the further polypeptide has protease activity, and optionally further medical cleaning components,
b) Subjecting at least part of the medical device to the wash liquor of step a),
c) Optionally, subjecting the medical device to rinsing,
d) Optionally, subjecting the medical device to drying,
e) Optionally, subjecting the medical device to sterile packaging, and
wherein the wash liquor releases and/or removes at least 50% of the organic soil present on the part of the medical device subjected to the wash liquor. In a more preferred embodiment at least 60% of the organic soil is released and/or removed, more preferably at least 70% of the organic soil is released and/or removed, even more preferably at least 80% of the organic soil is released and/or removed, most preferably at least 90% of the organic soil is released and/or removed, such as at least 95% of the organic soil is released and/or removed.

In another preferred embodiment, the invention relates to a method for cleaning a medical device comprising the steps of:
a) Providing a wash liquor having a pH in the range of 6.0-10.0, comprising a polypeptide having a sequence identity to the at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to any of the polypeptides shown in SEQ ID NOs: 1, 2, 3, 4, 5, 6, 7, 8, 9 or 10, wherein the polypeptide has DNase activity, and comprising a further polypeptide having a sequence identity to the polypeptide shown in SEQ ID NO: 12 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, wherein the further polypeptide has protease activity, and optionally further medical cleaning components,
b) Subjecting at least part of the medical device to the wash liquor of step a),
c) Optionally, subjecting the medical device to rinsing,
d) Optionally, subjecting the medical device to drying,
e) Optionally, subjecting the medical device to sterile packaging, and
wherein the wash liquor releases and/or removes at least 50% of the organic soil present on the part of the medical device subjected to the wash liquor. In a more preferred embodiment at least 60% of the organic soil is released and/or removed, more preferably at least 70% of the organic soil is released and/or removed, even more preferably at least 80% of the organic soil is released and/or removed, most preferably at least 90% of the organic soil is released and/or removed, such as at least 95% of the organic soil is released and/or removed.

In another preferred embodiment, the invention relates to a method for cleaning a medical device comprising the steps of:
a) Providing a wash liquor having a pH in the range of 6.0-10.0, comprising a polypeptide having a sequence identity to the at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to any of the polypeptides shown in SEQ ID NOs: 1, 2, 3, 4, 5, 6, 7, 8, 9 or 10, wherein the polypeptide has DNase activity, and comprising a further polypeptide having a sequence identity to the polypeptide shown in SEQ ID NO: 13 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, wherein the further polypeptide has protease activity, and optionally further medical cleaning components,
b) Subjecting at least part of the medical device to the wash liquor of step a),
c) Optionally, subjecting the medical device to rinsing,
d) Optionally, subjecting the medical device to drying,
e) Optionally, subjecting the medical device to sterile packaging, and
wherein the wash liquor releases and/or removes at least 50% of the organic soil present on the part of the medical device subjected to the wash liquor. In a more preferred embodiment at least 60% of the organic soil is released and/or removed, more preferably at least 70% of the organic soil is released and/or removed, even more preferably at least 80% of the organic soil is released and/or removed, most preferably at least 90% of the organic soil is released and/or removed, such as at least 95% of the organic soil is released and/or removed.

In another preferred embodiment, the invention relates to a method for cleaning a medical device comprising the steps of:
a) Providing a wash liquor having a pH in the range of 6.0-10.0, comprising a polypeptide selected from the group consisting of polypeptides comprising SEQ ID NOs: 1, 2, 3, 4, 5, 6, 7, 8, 9 and 10, and optionally further cleaning components,
b) Subjecting at least part of the medical device to the wash liquor of step a),
c) Optionally, subjecting the medical device to rinsing,
d) Optionally, subjecting the medical device to drying,
e) Optionally, subjecting the medical device to sterile packaging, and
wherein the wash liquor releases and/or removes at least 50% of the organic soil present on the part of the medical device subjected to the wash liquor. In a more preferred embodiment at least 60% of the organic soil is released and/or removed, more preferably at least 70% of the organic soil is released and/or removed, even more preferably at least 80% of the organic soil is released and/or removed, most preferably at least 90% of the organic soil is released and/or removed, such as at least 95% of the organic soil is released and/or removed.

In a further aspect, the invention concerns a medical cleaning composition comprising a polypeptide having DNase activity, wherein the composition has a pH in the range of 6.0-10.0.

The polypeptide having DNase activity can e.g. be obtained from Bacillus or Aspergillus. In a preferred embodiment, the polypeptide having DNase activity is obtained from Bacillus cibi or P. aeruginosa. The polypeptide having DNase activity may be used in the wash liquor or in the medical cleaning composition.

One embodiment relates to a medical cleaning composition comprising a polypeptide having DNase activity, wherein the composition preferably has a pH in the range of 6.0-10.0.

In one embodiment, the polypeptides having DNase activity have at least 20%, e.g., at least 40%, at least 50%, at least 60%, at least 70%, at least 80%, at least 90%, at least 95%, or at least 100% of the DNase activity of the mature polypeptide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10.

In one embodiment, the polypeptides having DNase activity have improved DNase activity, e.g. such that the DNase activity of the polypeptide is at least 105%, e.g., at least 110%, at least 120%, at least 130%, at least 140%, at least 160%, at least 170%, at least 180%, or at least 200% with reference to the DNase activity of the mature polypeptide of any one of SEQ ID NOs: 1-10.

In a preferred embodiment of the invention, the polypeptide having DNase activity is selected from the group consisting of:
a) a polypeptide having at least 60%, at least 70%, at least 80%, at least 90%, at least 95%, or at least 100% sequence identity to the polypeptide shown in SEQ ID NO: 1,
b) a polypeptide having at least 60%, at least 70%, at least 80%, at least 90%, at least 95%, or at least 100% sequence identity to the polypeptide shown in SEQ ID NO: 2,
c) a polypeptide having at least 60%, at least 70%, at least 80%, at least 90%, at least 95%, or at least 100% sequence identity to the polypeptide shown in SEQ ID NO: 3,
d) a polypeptide having at least 60%, at least 70%, at least 80%, at least 90%, at least 95%, or at least 100% sequence identity to the polypeptide shown in SEQ ID NO: 4,
e) a polypeptide having at least 60%, at least 70%, at least 80%, at least 90%, at least 95%, or at least 100% sequence identity to the polypeptide shown in SEQ ID NO: 5,
f) a polypeptide having at least 60%, at least 70%, at least 80%, at least 90%, at least 95%, or at least 100% sequence identity to the polypeptide shown in SEQ ID NO: 6,
g) a polypeptide having at least 60%, at least 70%, at least 80%, at least 90%, at least 95%, or at least 100% sequence identity to the polypeptide shown in SEQ ID NO: 7,
h) a polypeptide having at least 60%, at least 70%, at least 80%, at least 90%, at least 95%, or at least 100% sequence identity to the polypeptide shown in SEQ ID NO: 8,
i) a polypeptide having at least 60%, at least 70%, at least 80%, at least 90%, at least 95%, or at least 100% sequence identity to the polypeptide shown in SEQ ID NO: 9 and
j) a polypeptide having at least 60%, at least 70%, at least 80%, at least 90%, at least 95%, or at least 100% sequence identity to the polypeptide shown in SEQ ID NO: 10.

In a preferred embodiment, the present invention relates to a method for cleaning a medical device using a polypeptide having DNase activity, wherein the polypeptide has a sequence identity to the polypeptide of SEQ ID NO: 1 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In one aspect, the polypeptide differs by up to 10 amino acids, e.g., 1, 2, 3, 4, 5, 6, 7, 8, 9, or 10, from the mature polypeptide of SEQ ID NO: 1.

In a preferred embodiment, the present invention relates to a method for cleaning a medical device using a polypeptide having DNase activity, wherein the polypeptide has a sequence identity to the polypeptide of SEQ ID NO: 1 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In one aspect, the polypeptide differs by up to 10 amino acids, e.g., 1, 2, 3, 4, 5, 6, 7, 8, 9, or 10, from the mature polypeptide of SEQ ID NO: 2.

In a preferred embodiment, the present invention relates to a method for cleaning a medical device using a polypeptide having DNase activity, wherein the polypeptide has a sequence identity to the polypeptide of SEQ ID NO: 1 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In one aspect, the polypeptide differs by up to 10 amino acids, e.g., 1, 2, 3, 4, 5, 6, 7, 8, 9, or 10, from the mature polypeptide of SEQ ID NO: 3.

In a preferred embodiment, the present invention relates to a method for cleaning a medical device using a polypeptide having DNase activity, wherein the polypeptide has a sequence identity to the polypeptide of SEQ ID NO: 1 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In one aspect, the polypeptide differs by up to 10 amino acids, e.g., 1, 2, 3, 4, 5, 6, 7, 8, 9, or 10, from the mature polypeptide of SEQ ID NO: 4.

In a preferred embodiment, the present invention relates to a method for cleaning a medical device using a polypeptide having DNase activity, wherein the polypeptide has a sequence identity to the polypeptide of SEQ ID NO: 1 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In one aspect, the polypeptide differs by up to 10 amino acids, e.g., 1, 2, 3, 4, 5, 6, 7, 8, 9, or 10, from the mature polypeptide of SEQ ID NO: 5.

In a preferred embodiment, the present invention relates to a method for cleaning a medical device using a polypeptide having DNase activity, wherein the polypeptide has a sequence identity to the polypeptide of SEQ ID NO: 1 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In one aspect, the polypeptide differs by up to 10 amino acids, e.g., 1, 2, 3, 4, 5, 6, 7, 8, 9, or 10, from the mature polypeptide of SEQ ID NO: 6.

In a preferred embodiment, the present invention relates to a method for cleaning a medical device using a polypeptide having DNase activity, wherein the polypeptide has a sequence identity to the polypeptide of SEQ ID NO: 1 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In one aspect, the polypeptide differs by up to 10 amino acids, e.g., 1, 2, 3, 4, 5, 6, 7, 8, 9, or 10, from the mature polypeptide of SEQ ID NO: 7.

In a preferred embodiment, the present invention relates to a method for cleaning a medical device using a polypeptide having DNase activity, wherein the polypeptide has a sequence identity to the polypeptide of SEQ ID NO: 1 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In one aspect, the polypeptide differs by up to 10 amino acids, e.g., 1, 2, 3, 4, 5, 6, 7, 8, 9, or 10, from the mature polypeptide of SEQ ID NO: 8.

In a preferred embodiment, the present invention relates to a method for cleaning a medical device using a polypeptide having DNase activity, wherein the polypeptide has a sequence identity to the polypeptide of SEQ ID NO: 1 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In one aspect, the polypeptide differs by up to 10 amino acids, e.g., 1, 2, 3, 4, 5, 6, 7, 8, 9, or 10, from the mature polypeptide of SEQ ID NO: 9.

In a preferred embodiment, the present invention relates to a method for cleaning a medical device using a polypeptide having DNase activity, wherein the polypeptide has a sequence identity to the polypeptide of SEQ ID NO: 1 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In one aspect, the polypeptide differs by up to 10 amino acids, e.g., 1, 2, 3, 4, 5, 6, 7, 8, 9, or 10, from the mature polypeptide of SEQ ID NO: 10.

In a preferred embodiment, the present invention relates to a method for cleaning a medical device comprising the steps of:
a) Providing a wash liquor having a pH in the range of 6.0-10.0, comprising a polypeptide having DNase activity and optionally further cleaning components,
b) Subjecting at least part of the medical device to the wash liquor of step a),
c) Optionally, subjecting the medical device to rinsing,
d) Optionally, subjecting the medical device to drying, and
e) Optionally, subjecting the medical device to sterile packaging, wherein the polypeptide having DNase activity is
   i. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 1;
   ii. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 2;
   iii. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 3;
   iv. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 4,
   v. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 5,
   vi. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 6,
   vii. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 7,
   viii. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 8,
   ix. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 9; or
   x. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 10.

One embodiment relates to a medical cleaning composition further comprising a polypeptide having protease activity, wherein the composition preferably has a pH in the range of 6.0-10.0.

In a preferred embodiment, the medical cleaning composition and the method of the invention comprises a protease.

The protease enzymes for use in the present invention include those of bacterial, fungal, plant, viral or animal origin e.g. vegetable or microbial origin. Microbial origin is preferred. Chemically modified or protein engineered mutants are included.

Preferably, the protease is a subtilisin. In the context of the present invention, the subtilisin enzyme family (EC 3.4.21.62) shall be understood as described by Siezen et al., Protein Engng. 4 (1991) 719-737 and Siezen et al. Protein Science 6 (1997) 501-523. As described therein, the subtilisin family may be divided into 3 sub-groups, i.e. I-S1 (“true” subtilisins), I-S2 (highly alkaline proteases) and intracellular subtilisins.

Examples of subtilisins are those derived from Bacillus such as subtilisin lentus, Bacillus lentus, subtilisin Novo, subtilisin Carlsberg, Bacillus licheniformis, subtilisin BPN′, subtilisin 309, subtilisin 147 and subtilisin 168 described in WO 89/06279 and protease PD138 (WO 93/18140). Additional examples are described in WO 98/020115, WO 01/44452, WO 01/58275, WO 01/58276, WO 03/006602 and WO 04/099401.

Examples of useful variants are described in WO 92/19729, WO 98/20115, WO 98/20116, and WO 98/34946, especially the variants with substitutions in one or more of the following positions: 3, 4, 9, 15, 27, 36, 57, 68, 76, 87, 95, 96, 97, 98, 99, 100, 101, 102, 103, 104, 106, 118, 120, 123, 128, 129, 130, 160, 167, 170, 194, 195, 199, 205, 206, 217, 218, 222, 224, 232, 235, 236, 245, 248, 252 and 274 using the BPN′ numbering. More preferred the subtilase variants may comprise the mutations: S3T, V4I, S9R, A15T, K27R, *36D, V68A, N76D, N87S,R, *97E, A98S, S99G,D,A, S99AD, S101G,M,R S103A, V104I,Y,N, S106A, G118V,R, H120D,N, N123S, S128L, P129Q, S130A, G160D, Y167A, R170S, A194P, G195E, V199M, V205I, L217D, N218D, M222S, A232V, K235L, Q236H, Q245R, N252K, T274A (using BPN′ numbering).

Examples of commercially available subtilisins include Kannase™, Everlase™, Primase, Duralase, Esperase™, Alcalase™, Durazym™, Savinase™, Ovozyme™, Liquanase™, Coronase™, Polarzyme™, Pyrase™, and Clear-Lens™ Pro; Blaze™ (all available from Novozymes A/S). Other commercially available proteases include Ronozyme™ Pro, Maxatase™, Maxacal™, Maxapem™, Opticlean™, Properase™, Purafect™, Purafect Ox™, Purafact Prime™, Excellase™, FN2™, FN3™ and FN4™ (available from Dupont).

In a preferred embodiment, the present invention relates to a method for cleaning a medical device, wherein the method comprises use of a polypeptide having protease activity and having a sequence identity to the polypeptide of SEQ ID NO: 11 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In one aspect, the polypeptide differs by up to 10 amino acids, e.g., 1, 2, 3, 4, 5, 6, 7, 8, 9, or 10, from the mature polypeptide of SEQ ID NO: 11.

In a preferred embodiment, the present invention relates to a method for cleaning a medical device, wherein the method comprises use of a polypeptide having protease activity and having a sequence identity to the polypeptide shown in SEQ ID NO: 12 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In one aspect, the polypeptide differs by up to 10 amino acids, e.g., 1, 2, 3, 4, 5, 6, 7, 8, 9, or 10, from the mature polypeptide of SEQ ID NO: 12.

In a preferred embodiment, the present invention relates to a method for cleaning a medical device comprising the steps of:
a) Providing a wash liquor having a pH in the range of 6.0-10.0, comprising a polypeptide having protease activity and optionally further cleaning components,
b) Subjecting at least part of the medical device to the wash liquor of step a),
c) Optionally, subjecting the medical device to rinsing,
d) Optionally, subjecting the medical device to drying, and
e) Optionally, subjecting the medical device to sterile packaging, wherein the polypeptide having protease activity is
   i. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 11 or
   ii. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 12.

In a preferred embodiment, a mannanase is used in the method of the invention or is comprised in the medical cleaning composition of the invention.

One embodiment relates to a medical cleaning composition comprising a polypeptide having mannanase activity, wherein the composition preferably has a pH in the range of 6.0-10.0.

In a preferred embodiment, the medical cleaning composition and the method of the invention comprises a mannanase.

The mannanase may be an alkaline mannanase of Family 5 or 26. It may be a wild type from Bacillus or Humicola, particularly B. agaradhaerens, B. licheniformis, B. halodurans, B. clausii, or H. insolens. Suitable mannanases are described in WO 99/064619. A commercially available mannanase is Mannaway™ (Novozymes A/S).

In a preferred embodiment, the present invention relates to a method for cleaning a medical device comprising use of a polypeptide having mannanase and having a sequence identity to the polypeptide shown in SEQ ID NO: 13 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In one aspect, the polypeptide differs by up to 10 amino acids, e.g., 1, 2, 3, 4, 5, 6, 7, 8, 9, or 10, from the mature polypeptide of SEQ ID NO: 13.

In a preferred embodiment, the present invention relates to a method for cleaning a medical device comprising the steps of:
a) Providing a wash liquor having a pH in the range of 6.0-10.0, comprising a polypeptide having mannanase activity and optionally further cleaning components,
b) Subjecting at least part of the medical device to the wash liquor of step a),
c) Optionally, subjecting the medical device to rinsing,
d) Optionally, subjecting the medical device to drying, and
e) Optionally, subjecting the medical device to sterile packaging,
wherein the polypeptide having mannase activity is a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 13.

In a preferred embodiment, the present invention relates to a method for cleaning a medical device comprising the steps of:
a) Providing a wash liquor having a pH in the range of 6.0-10.0, comprising a polypeptide having DNase activity and optionally further cleaning components,
b) Subjecting at least part of the medical device to the wash liquor of step a),
c) Optionally, subjecting the medical device to rinsing,
d) Optionally, subjecting the medical device to drying, and
e) Optionally, subjecting the medical device to sterile packaging,
wherein the polypeptide having DNase activity is a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 1, and the wash liquor further comprises a polypeptide having protease activity and having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 11.

In a preferred embodiment, the present invention relates to a method for cleaning a medical device comprising the steps of:
a) Providing a wash liquor having a pH in the range of 6.0-10.0, comprising a polypeptide having DNase activity and optionally further cleaning components,
b) Subjecting at least part of the medical device to the wash liquor of step a),
c) Optionally, subjecting the medical device to rinsing,
d) Optionally, subjecting the medical device to drying, and
e) Optionally, subjecting the medical device to sterile packaging,
wherein the polypeptide having DNase activity is a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 1, and the wash liquor further comprises a polypeptide having protease activity and having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 12.

In a preferred embodiment, the present invention relates to a method for cleaning a medical device comprising the steps of:
a) Providing a wash liquor having a pH in the range of 6.0-10.0, comprising a polypeptide having DNase activity and optionally further cleaning components,
b) Subjecting at least part of the medical device to the wash liquor of step a),
c) Optionally, subjecting the medical device to rinsing,
d) Optionally, subjecting the medical device to drying, and
e) Optionally, subjecting the medical device to sterile packaging,
wherein the polypeptide having DNase activity is a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 1, and the wash liquor further comprises a polypeptide having mannanase activity and having at least 60% sequence,at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 13.

In a preferred embodiment, the present invention relates to a method for cleaning a medical device comprising the steps of:
a) Providing a wash liquor having a pH in the range of 6.0-10.0, comprising a polypeptide having DNase activity and optionally further cleaning components,
b) Subjecting at least part of the medical device to the wash liquor of step a),
c) Optionally, subjecting the medical device to rinsing,
d) Optionally, subjecting the medical device to drying, and
e) Optionally, subjecting the medical device to sterile packaging,
wherein the polypeptide having DNase activity is a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 8, and the wash liquor further comprises a polypeptide having protease activity and having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 11.

In a preferred embodiment, the present invention relates to a method for cleaning a medical device comprising the steps of:
a) Providing a wash liquor having a pH in the range of 6.0-10.0, comprising a polypeptide having DNase activity and optionally further cleaning components,
b) Subjecting at least part of the medical device to the wash liquor of step a),
c) Optionally, subjecting the medical device to rinsing,
d) Optionally, subjecting the medical device to drying, and
e) Optionally, subjecting the medical device to sterile packaging,
wherein the polypeptide having DNase activity is a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 8, and the wash liquor further comprises a polypeptide having protease activity and having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 12.

In a preferred embodiment, the present invention relates to a method for cleaning a medical device comprising the steps of:
a) Providing a wash liquor having a pH in the range of 6.0-10.0, comprising a polypeptide having DNase activity and optionally further cleaning components,
b) Subjecting at least part of the medical device to the wash liquor of step a),
c) Optionally, subjecting the medical device to rinsing,
d) Optionally, subjecting the medical device to drying, and
e) Optionally, subjecting the medical device to sterile packaging,
wherein the polypeptide having DNase activity is a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 8, and the wash liquor further comprises a polypeptide having mannanase activity and having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 13.

In a preferred embodiment, the present invention relates to a method for cleaning a medical device comprising the steps of:
a) Providing a wash liquor having a pH in the range of 6.0-10.0, comprising a polypeptide having DNase activity and optionally further cleaning components,
b) Subjecting at least part of the medical device to the wash liquor of step a),
c) Optionally, subjecting the medical device to rinsing,
d) Optionally, subjecting the medical device to drying, and
e) Optionally, subjecting the medical device to sterile packaging,
wherein the polypeptide having DNase activity is a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NOs: 1, 2, 3, 4, 5, 6, 7, 8, 9 or 10 and the wash liquor further comprises a polypeptide having protease activity and having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 11.

In a preferred embodiment, the present invention relates to a method for cleaning a medical device comprising the steps of:
a) Providing a wash liquor having a pH in the range of 6.0-10.0, comprising a polypeptide having DNase activity and optionally further cleaning components,
b) Subjecting at least part of the medical device to the wash liquor of step a),
c) Optionally, subjecting the medical device to rinsing,
d) Optionally, subjecting the medical device to drying, and
e) Optionally, subjecting the medical device to sterile packaging,
wherein the polypeptide having DNase activity is a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NOs: 1, 2, 3, 4, 5, 6, 7, 8, 9 or 10 and the wash liquor further comprises a polypeptide having protease activity and having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 12.

In a preferred embodiment, the present invention relates to a method for cleaning a medical device comprising the steps of:
a) Providing a wash liquor having a pH in the range of 6.0-10.0, comprising a polypeptide having DNase activity and optionally further cleaning components,
b) Subjecting at least part of the medical device to the wash liquor of step a),
c) Optionally, subjecting the medical device to rinsing,
d) Optionally, subjecting the medical device to drying, and
e) Optionally, subjecting the medical device to sterile packaging,
wherein the polypeptide having DNase activity is a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NOs: 1, 2, 3, 4, 5, 6, 7, 8, 9 or 10 and the wash liquor further comprises a polypeptide having mannanase activity and having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 13.

In a preferred embodiment, the present invention relates to a method for cleaning a medical device comprising the steps of:
a) Providing a wash liquor having a pH in the range of 6.0-10.0, comprising a polypeptide having DNase activity and optionally further cleaning components,
b) Subjecting at least part of the medical device to the wash liquor of step a),
c) Optionally, subjecting the medical device to rinsing,
d) Optionally, subjecting the medical device to drying, and
e) Optionally, subjecting the medical device to sterile packaging,
wherein the polypeptide having DNase activity is a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 1, 2, 3, 4, 5, 6, 7, 8, 9 or 10 and the wash liquor further comprises a polypeptide having protease activity and having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 11, and a polypeptide having mannanase activity and having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 13.

In a preferred embodiment, the present invention relates to a method for cleaning a medical device comprising the steps of:
a) Providing a wash liquor having a pH in the range of 6.0-10.0, comprising a polypeptide having DNase activity and optionally further cleaning components,
b) Subjecting at least part of the medical device to the wash liquor of step a),
c) Optionally, subjecting the medical device to rinsing,
d) Optionally, subjecting the medical device to drying, and
e) Optionally, subjecting the medical device to sterile packaging,
wherein the polypeptide having DNase activity is a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 1, 2, 3, 4, 5, 6, 7, 8, 9 or 10 and the wash liquor further comprises a polypeptide having protease activity and having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 12, and a polypeptide having mannanase activity and having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 13.

In a preferred embodiment, the present invention relates to a method for cleaning a medical device, wherein at least a part of the medical device comprises organic soil comprising one or more microorganisms, which part has been subjected to a liquid solution comprising peracetic acid having a pH of about 3, such as about 3.0, whereafter no live microorganisms are comprised in the organic soil, the method comprising the steps of:
a) Providing a wash liquor having a pH in the range of 6.0-10.0, comprising a polypeptide having DNase activity and optionally further cleaning components,
b) Subjecting at least part of the medical device to the wash liquor of step a),
c) Subjecting the medical device to rinsing,
d) Optionally, subjecting the medical device to drying, and
e) Optionally, subjecting the medical device to sterile packaging, preferably wherein the polypeptide having DNase activity is:
   i. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 1;
   ii. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 2;
   iii. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 3;
   iv. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 4.
   v. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 5,
   vi. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 6,
   vii. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 7,
   viii. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 8,
   ix. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 9, or
   x. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 10.

The microorganisms in the organic soil may comprise Pseudomonas aeruginosa.

In a preferred embodiment, the present invention relates to a method for cleaning a medical device, wherein at least a part of the medical device comprises organic soil comprising one or more microorganisms, which part has been subjected to a liquid solution comprising peracetic acid having a pH of about 3, such as about 3.0, whereafter no live microorganisms are comprised in the organic soil, the method comprising the steps of:
a) Providing a wash liquor having a pH in the range of 6.0-10.0, comprising a polypeptide having DNase activity and optionally further cleaning components,
b) Subjecting at least part of the medical device to the wash liquor of step a),
c) Subjecting the medical device to rinsing,
d) Optionally, subjecting the medical device to drying, and
e) Optionally, subjecting the medical device to sterile packaging, wherein the wash liquor further comprises a polypeptide having protease activity, wherein the polypeptide having protease activity is:
   i. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 11, or
   ii. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 12.

The microorganisms in the organic soil may comprise Pseudomonas aeruginosa.

In a preferred embodiment, the present invention relates to a method for cleaning a medical device, wherein at least a part of the medical device comprises organic soil comprising one or more microorganisms, which part has been subjected to a liquid solution comprising peracetic acid having a pH of about 3, such as about 3.0, whereafter no live microorganisms are comprised in the organic soil, the method comprising the steps of:
a) Providing a wash liquor having a pH in the range of 6.0-10.0, comprising a polypeptide having DNase activity and optionally further cleaning components,
b) Subjecting at least part of the medical device to the wash liquor of step a),
c) Subjecting the medical device to rinsing,
d) Optionally, subjecting the medical device to drying, and
e) Optionally, subjecting the medical device to sterile packaging, wherein the wash liquor further comprises a polypeptide having mannanase activity, wherein the polypeptide having mannanase activity is:
   i. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 13.

The microorganisms in the organic soil may comprise Pseudomonas aeruginosa.

In a preferred embodiment, the present invention relates to a method for cleaning a medical device, wherein at least a part of the medical device comprises organic soil comprising one or more microorganisms, which part has been subjected to a liquid solution comprising peracetic acid having a pH of about 3, such as about 3.0, whereafter no live microorganisms are comprised in the organic soil, the method comprising the steps of:
a) Providing a wash liquor having a pH in the range of 6.0-10.0, comprising a polypeptide having DNase activity and optionally further cleaning components,
b) Subjecting at least part of the medical device to the wash liquor of step a),
c) Subjecting the medical device to rinsing,
d) Optionally, subjecting the medical device to drying, and
e) Optionally, subjecting the medical device to sterile packaging, wherein the polypeptide having DNase activity is:
   i. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 1;
   ii. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 2;
   iii. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 3;
   iv. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 4;
   v. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 5;
   vi. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 6;
   vii. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 7;
   viii. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 8;
   ix. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 9, or
   x. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 10,
wherein the wash liquor further comprises a polypeptide having protease activity, and wherein the polypeptide having protease activity is a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 11.

The microorganisms in the organic soil may comprise Pseudomonas aeruginosa.

In a preferred embodiment, the present invention relates to a method for cleaning a medical device, wherein at least a part of the medical device comprises organic soil comprising one or more microorganisms, which part has been subjected to a liquid solution comprising peracetic acid having a pH of about 3, such as about 3.0, whereafter no live microorganisms are comprised in the organic soil, the method comprising the steps of:
a) Providing a wash liquor having a pH in the range of 6.0-10.0, comprising a polypeptide having DNase activity and optionally further cleaning components,
b) Subjecting at least part of the medical device to the wash liquor of step a),
c) Subjecting the medical device to rinsing,
d) Optionally, subjecting the medical device to drying, and
e) Optionally, subjecting the medical device to sterile packaging, wherein the polypeptide having DNase activity is:
   i. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 1;
   ii. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 2;
   iii. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 3;
   iv. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 4;
   v. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 5;
   vi. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 6;
   vii. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 7;
   viii. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 8;
   ix. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 9, or
   x. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 10,
wherein the wash liquor further comprises a polypeptide having protease activity, and wherein the polypeptide having protease activity is a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 12.

The microorganisms in the organic soil may comprise Pseudomonas aeruginosa.

In a preferred embodiment, the present invention relates to a method for cleaning a medical device, wherein at least a part of the medical device comprises organic soil comprising one or more microorganisms, which part has been subjected to a liquid solution comprising peracetic acid having a pH of about 3, such as about 3.0, whereafter no live microorganisms are comprised in the organic soil, the method comprising the steps of:
a) Providing a wash liquor having a pH in the range of 6.0-10.0, comprising a polypeptide having DNase activity and optionally further cleaning components,
b) Subjecting at least part of the medical device to the wash liquor of step a),
c) Subjecting the medical device to rinsing,
d) Optionally, subjecting the medical device to drying, and
e) Optionally, subjecting the medical device to sterile packaging, wherein the polypeptide having DNase activity is:
   i. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 1;
   ii. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 2;
   iii. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 3;
   iv. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 4;
   v. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 5;
   vi. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 6;
   vii. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 7;
   viii. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 8;
   ix. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 9, or
   x. a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 10,
wherein the wash liquor further comprises a polypeptide having mannanse activity, and wherein the polypeptide having mannanase activity is a polypeptide having at least 60% sequence, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide shown in SEQ ID NO: 13.

The microorganisms in the organic soil may comprise Pseudomonas aeruginosa.

In one aspect of the invention, a polypeptide having DNase activity is used for releasing and/or removing organic soil from a medical device.

The device may be indwelling medical device characterized in that at least a portion of a patient-contactable surface of the device is coated with a composition comprising a polypeptide having DNase activity.

In one aspect of the invention, a polypeptide having DNase activity and a polypeptide having protease activity are used for releasing and/or removing organic soil from a medical device.

The device may be indwelling medical device characterized in that at least a portion of a patient-contactable surface of the device is coated with a composition comprising a polypeptide having DNase activity and a polypeptide having protease activity.

In one aspect of the invention, a polypeptide having DNase activity and a polypeptide having mannanase activity are used for releasing and/or removing organic soil from a medical device.

The device may be indwelling medical device characterized in that at least a portion of a patient-contactable surface of the device is coated with a composition comprising a polypeptide having DNase activity and a polypeptide having mannanase activity.

In one aspect of the invention, a polypeptide having DNase activity, a polypeptide having protease activity and a polypeptide having mannanase activity are used for releasing and/or removing organic soil from a medical device.

The device may be indwelling medical device characterized in that at least a portion of a patient-contactable surface of the device is coated with a composition comprising a polypeptide having DNase activity, a polypeptide having protease activity and a polypeptide having mannanase activity.

The medical device to be cleaned by the methods and compositions disclosed herein may be any appropriate medical device, including but not limited to a catheter such as a central venous catheter, intravascular catheter, urinary catheter, Hickman catheter, peritoneal dialysis catheter, endrotracheal catheter, or wherein the device is a mechanical heart valve, a cardiac pacemaker, an arteriovenous shunt, a scleral buckle, a prosthetic joint, a tympanostomy tube, a tracheostomy tube, a voice prosthetic, a penile prosthetic, an artificial urinary sphincter, a synthetic pubovaginal sling, a surgical suture, a bone anchor, a bone screw, an intraocular lens, a contact lens, an intrauterine device, an aortofemoral graft, a vascular graft, a needle, a Luer-Lok connector, a needleless connector, surgical instrument, diagnostic instruments, trays, pans, holders, racks, forceps, scissors, shears, saws (e.g. bone saws and their blades), hemostats, knives, chisels, rongeurs, files, nippers, drills, drill bits, rasps, burrs, spreaders, breakers, elevators, clamps, needle holders, carriers, clips, hooks, gouges, curettes, retractors, straightener, punches, extractors, scoops, keratomes, spatulas, expressors, trocars, dilators, cages, glassware, tubing, catheters, cannulas, plugs, stents, endoscopes, arthoscopes and related equipment.

In one aspect, the invention concerns a medical device coated with a composition comprising a polypeptide having DNase activity, a polypeptide having protease activity and/or polypeptide having mannanse activity. The medical device may be characterized in that at least a portion of a patient-contactable surface of the device is coated with composition comprising a polypeptide having DNase activity, a polypeptide having protease activity and/or a polypeptide having mannanse activity.

The medical cleaning composition may be in the form of a bar, a homogenous tablet, a tablet having two or more layers, a pouch having one or more compartments, a regular or compact powder, a granule, a paste, a gel, a regular liquid, a compact liquid or concentrated liquid.

A liquid medical cleaning composition may be a pourable liquid, a pourable gel or a non-pourable gel. It may be isotropic or structured, preferably isotropic. It may be a formulation useful for washing in automatic washing machines or for hand washing.

The liquid medical cleaning composition may be aqueous, typically containing at least 20% by weight and up to 95% water, such as up to 70% water, up to 50% water, up to 40% water, up to 30% water, or up to 20% water. Other types of liquids, including without limitation, alkanols, amines, diols, ethers and polyols, may be included in an aqueous liquid detergent. An aqueous liquid detergent may contain from 0-30% organic solvent. A liquid medical cleaning composition may even be non-aqueous, wherein the water content is below 10%, preferably below 5%.

Medical cleaning components can be separated physically from each other by compartments in water dissolvable pouches. Thereby negative storage interaction between components can be avoided. Different dissolution profiles of each of the compartments can also give rise to delayed dissolution of selected components in the wash solution.

In addition to the polypeptides having DNase, protease or mannanase activity, the medical cleaning composition may comprise additional enzymes as well as medical cleaning components. The choice of additional components is within the skill of the artisan and includes conventional ingredients, including the exemplary non-limiting components set forth below. In a preferred embodiment the medical cleaning composition comprises additional enzymes.

In one embodiment, a lipase is used in the method of the invention or is comprised in the medical cleaning composition of the invention.

Suitable lipases and cutinases include those of bacterial or fungal origin. Chemically modified or protein engineered mutants are included. Examples include lipase from Thermomyces, e.g., from T. lanuginosus (previously named Humicola lanuginosa) as described in EP 258068 and EP 305216, cutinase from Humicola, e.g. H. insolens as described in WO 96/13580, a Pseudomonas lipase, e.g., from P. alcaligenes or P. pseudoalcaligenes (EP 218272), P. cepacia (EP 331376), P. stutzeri (GB 1372034), P. fluorescens, Pseudomonas sp. strain SD 705 (WO 95/06720 and WO 96/27002), P. wisconsinensis (WO 96/12012), a Bacillus lipase, e.g., from B. subtilis (Dartois et al., Biochemica et Biophysica Acta, (1993), 1131, 253-360), B. stearothermophilus (JP 64/744992) or B. pumilus (WO 91/16422), GDSL-type Streptomyces lipases (WO10/065455), cutinase from Magnaporthe grisea (WO10/107560), cutinase from Pseudomonas mendocina (US5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in WO 92/05249, WO 94/01541, EP 407225, EP 260105, WO 95/35381, WO 96/00292, WO 95/30744, WO 94/25578, WO 95/14783, WO 95/22615, WO 97/04079, WO 97/07202, WO 00/060063, WO 07/087508 and WO 09/109500.

Preferred commercially available lipase enzymes include Lipolase™, Lipolase Ultra™, and Lipex™; Lecitase™, Lipolex™; Lipoclean™, Lipoprime™ (Novozymes A/S). Other commercially available lipases include Lumafast (Genencor Int Inc); Lipomax (Gist-Brocades/Genencor Int Inc) and Bacillus sp lipase from Solvay.

In one embodiment, a carbohydrase is used in the method of the invention or is comprised in the medical cleaning composition of the invention.

A carbohydrase is a general term for enzymes that cleave carbohydrates. In general carbohydrases are named after the substrates they act on, for example amylases act on amylase and cellulases act on cellulose. Many carbohydrases have found use in cleaning and laundry applications, such as amylase, cellulase, pectinase, pectate lyase, mannanase, arabinase, galactanase and xylanase, and all these can be applied in the liquid cleaning composition.

In one embodiment, an amylase is used in the method of the invention or is comprised in the medical cleaning composition of the invention.

Suitable amylases include those of bacterial or fungal origin. Chemically modified or protein engineered mutants are included. Amylases include, for example, a-amylases obtained from Bacillus, e.g., a special strain of Bacillus licheniformis, described in more detail in GB 1,296,839.

Examples of suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193. Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, I201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:
M197T;
H156Y+A181T+N190F+A209V+Q264S; or
G48A+T49I+G107A+H156Y+A181T+N190F+I201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, I206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476. More preferred variants are those having a deletion in positions 181 and 182 or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:
N128C+K178L+T182G+Y305R+G475K;
N128C+K178L+T182G+F202Y+Y305R+D319T+G475K;
S125A+N128C+K178L+T182G+Y305R+G475K; or
S125A+N128C+T131I+T165I+K178L+T182G+Y305R+G475K wherein the variants are C-terminally truncated and optionally further comprises a substitution at position 243 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particularly preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Stainzyme™, Stainzyme Plus™, Amplify™, Resilience™, Everest™, Duramyl™, Termamyl™, Termamyl Ultra™; Natalase™, Fungamyl™ and BAN™ (Novozymes A/S), Rapidase™ and Purastar™/Effectenz™, Powerase™ and Preferenz S100 (from Genencor International Inc./DuPont).

In one embodiment, a lyase is used in the method of the invention or is comprised in the medical cleaning composition of the invention.

The lyase may be a pectate lyase derived from Bacillus, particularly B. licherniformis or B. agaradhaerens, or a variant derived of any of these, e.g. as described in US 6124127, WO 99/027083, WO 99/027084, WO 02/006442, WO 02/092741, WO 03/095638, Commercially available pectate lyases are XPect™, Pectawash™, and Pectaway™ (Novozymes A/S).

In one embodiment, a cellulase is used in the method of the invention or is comprised in the medical cleaning composition of the invention.

Suitable cellulases may be of bacterial or fungal origin. Chemically or genetically modified mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in US 4,435,307, US 5,648,263, US 5,691,178, US 5,776,757 and WO 89/09259.

Especially suitable cellulases are the alkaline or neutral cellulases having color care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, US 5,457,046, US 5,686,593, US

Commercially available cellulases include Carezyme™, Celluzyme™, Celluclean™, Celluclast™, Endolase™, Renozyme™, Whitezyme™ (Novozymes A/S); Clazinase™, Puradax, Puradax HA, and Puradax EG (available from Genencor) and KAC-500(B)™ (Kao Corporation).

In one embodiment, a peroxidase is used in the method of the invention or is comprised in the medical cleaning composition of the invention.

Suitable peroxidases are comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257.

The peroxidases also include a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions.

In an embodiment, the haloperoxidase is a chloroperoxidase. Preferably, the haloperoxidase is a vanadium haloperoxidase, i.e., a vanadate-containing haloperoxidase. In a preferred method, the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

In an preferred embodiment, the haloperoxidase is derivable from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 01/79459, Dendryphiella salina as described in WO 01/79458, Phaeotrichoconis crotalarie as described in WO 01/79461, or Geniculosporium sp. as described in WO 01/79460.

Suitable oxidases include, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5).

Preferred laccase enzymes are enzymes of microbial origin. The enzymes may be derived from plants, bacteria or fungi (including filamentous fungi and yeasts).

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885).

Suitable examples from bacteria include a laccase derivable from a strain of Bacillus.

A laccase derived from Coprinopsis or Myceliophthora is preferred; in particular a laccase derived from Coprinopsis cinerea, as disclosed in WO 97/08325; or from Myceliophthora thermophila, as disclosed in WO 95/33836.

In a preferred embodiment, a perhydrolase is used in the method of the invention or is comprised in the medical cleaning composition of the invention.

Suitable perhydrolases are capable of catalyzing a perhydrolysis reaction that results in the production of a peracid from a carboxylic acid ester (acyl) substrate in the presence of a source of peroxygen (e.g., hydrogen peroxide). While many enzymes perform this reaction at low levels, perhydrolases exhibit a high perhydrolysis:hydrolysis ratio, often greater than 1. Suitable perhydrolases may be of plant, bacterial or fungal origin. Chemically modified or protein engineered mutants are included.

Examples of useful perhydrolases include naturally occurring Mycobacterium perhydrolase enzymes, or variants thereof. An exemplary enzyme is derived from Mycobacterium smegmatis. Such enzyme, its enzymatic properties, its structure, and variants thereof, are described in WO 2005/056782, WO 2008/063400, US 2008/145353, and US2007167344.

In a preferred embodiment, the medical cleaning composition comprises at least one medical cleaning component.

The medical cleaning composition may comprise one or more wetting agents, which are surfactants, that may be anionic and/or cationic and/or non-ionic and/or semi-polar and/or zwitterionic, or a mixture thereof. In a particular embodiment, the medical cleaning composition includes a mixture of one or more nonionic surfactants and one or more anionic surfactants. The surfactant(s) is typically present at a level of from about 0% to 25% by weight, such as about 1% to about 25%, or about 3% to about 20%, or about 3% to about 10%. The surfactant(s) is chosen based on the desired cleaning application, and includes any conventional surfactant(s) known in the art. Any surfactant known in the art for use in cleaning compositions may be utilized.

When included therein the medical cleaning composition will usually contain from about 0% to about 5% by weight, such as from about 0% to about 4%, including from about 0% to about 3%, or from about 0% to about 2% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or soap, and combinations thereof.

When included therein the medical cleaning composition will usually contain from about 0.1% to about 10% by weight of a cationic surfactant. Non-limiting examples of cationic surfactants include alklydimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, and combinations thereof.

When included therein the medical cleaning composition will usually contain from about 5% to about 25% by weight of a non-ionic surfactant, for example from about 5% to about 20%, in particular from about 5% to about 15%, from about 5% to about 10%. Non-limiting examples of non-ionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), as well as products available under the trade names SPAN and TWEEN, and combinations thereof.

When included therein the medical cleaning composition will usually contain from about 0.1% to about 20% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, fatty acid alkanolamides and ethoxylated fatty acid alkanolamides, and combinations thereof.

When included therein the medical cleaning composition will usually contain from about 0.1% to about 10% by weight of a zwitterionic surfactant. Non-limiting examples of zwitterionic surfactants include betaine, alkyldimethylbetaine, sulfobetaine, and combinations thereof.

In one aspect of the invention, the wash liquor and/or the medical cleaning composition comprises a hydrotrope. A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants); however, the molecular structure of hydrotropes generally do not favor spontaneous self-aggregation, see for example review by Hodgdon and Kaler (2007), Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in cleaning compositions allow for example more concentrated formulations of surfactants (as in the process of compacting liquid cleaning compositions by removing water) without inducing undesired phenomena such as phase separation or high viscosity.

The medical cleaning composition may contain 0-5% by weight, such as about 0.5 to about 5%, or about 3% to about 5%, of a hydrotrope. Any hydrotrope known in the art for use in cleaning compositions may be utilized. Non-limiting examples of hydrotropes include sodium benzene sulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

In one aspect of the invention, the wash liquor and/or the medical cleaning composition comprises a detergent builder or co-builder.

The medical cleaning composition may contain about 0-65% by weight, such as about 5% to about 50% of a detergent builder or co-builder, or a mixture thereof. The builder and/or co-builder may particularly be a chelating agent that forms water-soluble complexes with Ca and Mg ions. Any builder and/or co-builder known in the art for use in laundry detergents may be utilized. Non-limiting examples of builders include citrates, zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as iminodiethanol), triethanolamine (TEA, also known as 2,2′,2″-nitrilotriethanol), and carboxymethyl inulin (CMI), and combinations thereof.

The medical cleaning composition may also contain 0-50% by weight, such as about 0.5% to about 10%, of a detergent co-builder, or a mixture thereof. The medical cleaning composition may include include a co-builder alone, or in combination with a builder, for example a citrate builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N, N-diacetic acid (α-ALDA), serine-N, N-diacetic acid (SEDA), isoserine-N, N-diacetic acid (ISDA), phenylalanine-N, N-diacetic acid (PHDA), anthranilic acid-N, N-diacetic acid (ANDA), sulfanilic acid-N, N-diacetic acid (SLDA), taurine-N, N-diacetic acid (TUDA) and sulfomethyl-N, N-diacetic acid (SMDA), N-(2-hydroxyethyl)-ethylidenediamine-N, N′, N′-triacetate (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, US 5977053.

In one aspect of the invention, the wash liquor and/or the medical cleaning composition comprises a polymer.

The medical cleaning composition may contain 0-10% by weight, such as 0.5-5%, 2-5%, 0.5-2% or 0.2-1% of a polymer. Any polymer known in the art for use in cleaning compositions may be utilized. The polymer may function as a co-builder as mentioned above, or may provide soil release, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575 and US 5,955,415. Salts of the above-mentioned polymers are also contemplated.

In one aspect of the invention, the wash liquor and/or the medical cleaning composition comprises a bleaching system.

Due to the incompatibility of the components there are still only few examples of liquid cleaning compositions combining bleach and enzymes (e.g., US 5,275,753 or WO 99/00478). The medical cleaning composition may contain 0-50% of a bleaching system. Any bleaching system known in the art for use in laundry cleaning compositions may be utilized. Suitable bleaching system components include bleaching catalysts, photobleaches, bleach activators, sources of hydrogen peroxide such as sodium percarbonate and sodium perborates, preformed peracids and mixtures thereof. Suitable preformed peracids include, but are not limited to, peroxycarboxylic acids and salts, percarbonic acids and salts, perimidic acids and salts, peroxymonosulfuric acids and salts, for example, Oxone (R), and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator. The term bleach activator is meant herein as a compound which reacts with peroxygen bleach like hydrogen peroxide to form a peracid. The peracid thus formed constitutes the activated bleach. Suitable bleach activators to be used herein include those belonging to the class of esters amides, imides or anhydrides. Suitable examples are tetracetylethylene diamine (TAED), sodium 4-[(3,5,5-trimethylhexanoyl)oxy]benzene sulfonate (ISONOBS), diperoxy dodecanoic acid, 4-(dodecanoyloxy)benzenesulfonate (LOBS), 4-(decanoyloxy)benzenesulfonate, 4-(decanoyloxy)benzoate (DOBS), 4-(nonanoyloxy)-benzenesulfonate (NOBS), and/or those disclosed in WO 98/17767. A particular family of bleach activators of interest was disclosed in EP624154 and particulary preferred in that family is acetyl triethyl citrate (ATC). ATC or a short chain triglyceride like triacetin has the advantage that it is environmentally friendly as it eventually degrades into citric acid and alcohol. Furthermore, acetyl triethyl citrate and triacetin has a good hydrolytical stability in the product upon storage and it is an efficient bleach activator. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst. In some embodiments the bleach component may be an organic catalyst selected from the group consisting of organic catalysts having the following formulae:

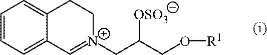

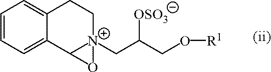

and mixtures thereof; wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, n-dodecyl, n-tetradecyl, n-hexadecyl, n-octadecyl, iso-nonyl, iso-decyl, iso-tridecyl and iso-pentadecyl. Other exemplary bleaching systems are described, e.g., in WO 2007/087258, WO 2007/087244, WO 2007/087259 and WO 2007/087242. Suitable photobleaches may for example be sulfonated zinc phthalocyanine.

In one aspect of the invention, the wash liquor and/or the medical cleaning composition comprises at least one polymer.

The detergent may contain 0.005-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or antifoaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(ethyleneglycol) or poly(ethylene oxide) (PEG or PEO), ethoxylated poly(ethyleneimine), (carboxymethyl)inulin (CMI), carboxylate polymers and polycarboxylates such as polyacrylates, maleic/acrylic acid copolymers, acrylate/styrene copolymers, poly(aspartic) acid, and lauryl methacrylate/acrylic acid copolymers, hydrophobically modified CMC (HM-CMC), silicones, copolymers of terephthalic acid and oligomeric glycols, copolymers of poly(ethylene terephthalate) and poly(oxyethene terephthalate) (PET-POET), poly(vinylpyrrolidone) (PVP), poly(vinylimidazole) (PVI), poly(vinylpyridine-N-oxide) (PVPO or PVPNO) and copoly(vinylimidazole/vinylpyrrolidone) (PVPVI). Suitable examples include PVP-K15, PVP-K30, ChromaBond S-400, ChromaBond S- 403E and Chromabond S-100 from Ashland Aqualon, and Sokalan® HP 165, Sokalan® HP 50 (Dispersing agent), Sokalan® HP 53 (Dispersing agent), Sokalan® HP 59 (Dispersing agent), Sokalan® HP 56 (dye transfer inhibitor), Sokalan® HP 66 K (dye transfer inhibitor) from BASF. Further exemplary polymers include sulfonated polycarboxylates, polyethylene oxide and polypropylene oxide (PEO-PPO) and diquaternium ethoxy sulfate. Particularly preferred polymer is ethoxylated homopolymer Sokalan® HP 20 from BASF, which helps to prevent redeposition of soil in the wash liquor. Further exemplary polymers include sulfonated polycarboxylates, ethylene oxide-propylene oxide copolymers (PEO-PPO), copolymers of PEG with and vinyl acetate, and diquaternium ethoxy sulfate or quaternized sulfated ethoxylated hexamethylenediamine. Other exemplary polymers are disclosed in, e.g., WO 2006/130575. Salts of the above-mentioned polymers are also contemplated.

The medical cleaning components can be separated physically from each other by compartments in water dissolvable pouches. Thereby negative storage interaction between components can be avoided. Different dissolution profiles of each of the compartments can also give rise to delayed dissolution of selected components in the wash solution.

The invention is further summarized in the following embodiments.
1. A method for cleaning a medical device comprising the steps of:
   a) Providing a wash liquor having a pH in the range of 6.0-10.0, comprising a polypeptide having DNase activity and optionally further cleaning components,
   b) Subjecting at least part of the medical device to the wash liquor of step a),
   c) Optionally, subjecting the medical device to rinsing,
   d) Optionally, subjecting the medical device to drying, and
   e) Optionally, subjecting the medical device to sterile packaging.
2. The method according to embodiment 1, wherein at least a part of the medical device comprises organic soil.
3. The method according to any of the preceding embodiments, wherein the organic soil comprises one or more microorganisms.
4. The method according to any of the preceding embodiments, wherein the microorganisms is selected from the group consisting of Acinetobacter sp., Aeromicrobium sp., Aspergillus versicolor, Brevundimonas sp., Enterobacter cloacae, Enterococcus faecalis, Escherichia coli, Klebsiella pneumoniae, Microbacterium sp., Micrococcus luteus, Pseudomonas sp., Staphylococcus aureus, Staphylococcus epidermidis, Stenotrophomonas sp., Bacillus sp., and Pseudomonas aeruginosa.
5. The method according to embodiment 4, wherein the microorganisms comprises Pseudomonas aeruginosa.
6. The method according to any of the preceding embodiments, wherein the wash liquor comprising a polypeptide having DNase activity releases and/or removes at least part of the organic soil present on the part of the medical device subjected to the wash liquor.
7. The method according to any of the preceding embodiments, wherein the medical device is disinfected before being subjected to the wash liquor.
8. The method according to any of the preceding embodiments, wherein substantially no live microorganisms are comprised in the organic soil.
9. The method according to embodiment 8, wherein at least part of the medical device is subjected to a liquid solution comprising peracetic acid having a pH of about 3, such as about 3.0, whereafter no live microorganisms are comprised in the organic soil.
10. The method according to any of the preceding embodiments, wherein at least 95% of the microorganisms comprised in the organic soil are dead.
11. The method according to any of the preceding embodiments, wherein the disinfection is performed by subjecting at least part of the medical device to a chemical or a biological disinfection composition or to radiation.
12. The method according to embodiment 11, wherein the disinfection is performed by subjecting at least part of the medical device to antimicrobial agents, antibiotics, fungicides, oxidizing agents, hydrogen peroxide, chlorine, acids, peroxy acids, peracetic acid, potassium permanganate, ultra-violet radiation (UV), ozon.
13. The method according to any of the preceding embodiments, wherein the pH of the wash liquor is in the range of 6.5-9.5, preferably in the range of 7.0-9.0, more preferably in the range of 7.5-8.5, such as a pH around 8.
14. The method according to any of the preceding embodiments, wherein the wash liquor comprises at least 0.001 ppm of the polypeptide having DNase activity, preferably at least 0.005 ppm of the polypeptide having DNase activity, more preferably at least 0.01 ppm of the polypeptide having DNase activity, even more preferably at least 0.05 ppm of the polypeptide having DNase activity or most preferably at least 1 ppm of the polypeptide having DNase activity.
15. The method according to any of the preceding embodiments, wherein the wash liquor comprises at most 100 ppm of the polypeptide having DNase activity, preferably at most 50 ppm of the polypeptide having DNase activity, more preferably at most 30 ppm of the polypeptide having DNase activity, even more preferably at most 20 of the polypeptide having DNase activity or most preferably at most 10 ppm of the polypeptide having DNase activity.
16. The method according to any of the preceding embodiments, wherein the wash liquor comprises in the range of 0.001-100 ppm of the polypeptide having DNase activity, preferably in the range of 0.005-50 ppm of the polypeptide having DNase activity, more preferably in the range of 0.01-30 ppm of the polypeptide having DNase activity, even more preferably in the range of 0.05-20 ppm of the polypeptide having DNase activity or most preferably in the range of 1-10 ppm more of the polypeptide having DNase activity.
17. The method according to any of the preceding embodiments, wherein the wash liquor further comprises one or more cleaning composition components, preferably selected from surfactants, builders, bleach components, polymers, dispersing agents and additional enzymes.
18. The method according to any of the preceding embodiments, wherein the wash liquor further comprises one or more enzymes selected from the group consisting of proteases, amylases, lipases, cutinases, cellulases, endoglucanases, xyloglucanases, pectinases, pectin lyases, xanthanases, peroxidases, haloperoxygenases, catalases, galactanase, mannanases, or any mixture thereof.
19. The method according to any of the preceding embodiments, wherein at least part of the medical device is subjected to the wash liquor by spraying, dipping or immersing.
20. The method according to any of the preceding embodiments, wherein the medical device is subjected to the wash liquor by immersing at least part of the medical device into the wash liquor.
21. The method according to embodiment 20, wherein at least part of the medical device is soaked in the wash liquor for a time sufficient to reduce and/or remove soil on the instrument, preferably for at least 1 minute.
22. The method according to any of the preceding embodiments, wherein the medical device is subjected to rinsing after being subjected to the wash liquor.
23. The method according to any of the preceding embodiments, wherein the medical device is subjected to drying after being subjected to the wash liquor.
24. The method according to any of the preceding embodiments, wherein the medical device is subjected to sterile packaging after being subjected to the wash liquor.
25. The method according to any of the preceding embodiments, wherein the wash liquor comprising a polypeptide having DNase activity releases and/or removes at least 50% of the organic soil present on the part of the medical device subjected to the wash liquor as measured in Example 2, preferably at least 60% of the organic soil is released and/or removed, more preferably at least 70% of the organic soil is released and/or removed, even more preferably at least 80% of the organic soil is released and/or removed, most preferably at least 90% of the organic soil is released and/or removed, such as at least 95% of the organic soil is released and/or removed.
26. The method according to any of the preceding embodiments, wherein the medical device is an indwelling device.
27. The method according to any of the preceding embodiments, wherein the polypeptide having DNase activity is of bacterial or fungal origin.
28. The method according to any of the preceding embodiments wherein the polypeptide having DNase activity is obtained from Bacillus or Aspergillus.
29. The method according to embodiment 27, wherein the polypeptide having DNase activity is obtained from Bacillus cibi or Aspergillus oryzae.
30. The method according to any of the preceding embodiments, wherein the polypeptide having DNase activity is selected from the group consisting of:
   a) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 1;
   b) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 2;
   c) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 3;
   d) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 4;
   e) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 5;
   f) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 6;
   g) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 7;
   h) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 8;
   i) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 9 and
   j) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 10.
31. A medical cleaning composition comprising a polypeptide having DNase activity, wherein the composition has a pH in the range of 6.0-10.0.
32. A medical cleaning composition according to embodiment 31, wherein the polypeptide is selected from the group consisting of:
   a) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 1;
   b) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 2;
   c) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 3;
   d) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 4;
   e) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 5;
   f) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 6;
   g) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 7;
   h) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 8;
   i) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 9 and
   j) a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 10.
33. The medical cleaning composition according to embodiment 32, wherein the composition further comprises one or more cleaning composition components, preferably selected from surfactants, builders, bleach components, polymers, dispersing agents and additional enzymes.
34. The medical cleaning composition according any of embodiments 31-33, wherein the medical cleaning composition further comprises one or more enzymes selected from the group consisting of proteases, amylases, lipases, cutinases, cellulases, endoglucanases, xyloglucanases, pectinases, pectin lyases, xanthanases, peroxidases, haloperoxygenases, catalases, galactanase, mannanases, or any mixture thereof.
35. The medical cleaning composition according to any of embodiments 31-34, wherein the composition comprises a protease and/or a mannanase, wherein the protease is:
   a) a polypeptide having 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 11;
   b) a polypeptide having 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 12, and wherein the mannanase is:
   c) a polypeptide having 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in sequence identity to the polypeptide shown in SEQ ID NO: 13.
36. The medical cleaning composition according any of embodiments 31-35 comprising 5-25% of a non-ionic surfactant and 0-5% of an anionic surfactant.
37. The medical cleaning composition according embodiment 36, wherein the composition comprises 5-20% of a non-ionic surfactant, preferably 5-15% or more preferably 5-10% of a non-ionic surfactant.
38. The medical cleaning composition according embodiments 36-37, wherein the composition comprises 0-4% of a anionic surfactant, preferably 0-3%, or more preferably 0-2% of a anionic surfactant.
39. The medical cleaning composition according to any of embodiments 31-38, wherein the composition is in the form of a bar, a homogenous tablet, a tablet having two or more layers, a pouch having one or more compartments, a regular or compact powder, a granule, a paste, a gel, a regular liquid, a compact liquid or concentrated liquid.
40. Use of a polypeptide having DNase activity for releasing and/or removing or organic soil from a medical device.
41. Use according to embodiment 40, wherein the polypeptide having DNase activity is used for coating at least part of a medical device.
42. Use according to any of embodiments 40-41, wherein the medical device is an indwelling device.
43. Use according to any of embodiments 40-42, wherein the medical device is selected from the group consisting of a catheter such as a central venous catheter, intravascular catheter, urinary catheter, Hickman catheter, peritoneal dialysis catheter, endrotracheal catheter, or wherein the device is a mechanical heart valve, a cardiac pacemaker, an arteriovenous shunt, a scleral buckle, a prosthetic joint, a tympanostomy tube, a tracheostomy tube, a voice prosthetic, a penile prosthetic, an artificial urinary sphincter, a synthetic pubovaginal sling, a surgical suture, a bone anchor, a bone screw, an intraocular lens, a contact lens, an intrauterine device, an aortofemoral graft, a vascular graft, a needle, a Luer-Lok connector, a needleless connector, surgical instrument, diagnostic instruments, trays, pans, holders, racks, forceps, scissors, shears, saws (e.g. bone saws and their blades), hemostats, knives, chisels, rongeurs, files, nippers, drills, drill bits, rasps, burrs, spreaders, breakers, elevators, clamps, needle holders, carriers, clips, hooks, gouges, curettes, retractors, straightener, punches, extractors, scoops, keratomes, spatulas, expressors, trocars, dilators, cages, glassware, tubing, catheters, cannulas, plugs, stents, endoscopes, arthoscopes and related equipment.
44. Medical device coated with a composition comprising a polypeptide having DNase activity.

### Cleaning Compositions

Any of the medical cleaning compositions described below may include one or more polypeptides having DNase activity and any number of additional enzymes. In general, the enzyme(s) should be compatible with the selected cleaning composition, (e.g., with respect to pH-optimum, compatibility with other enzymatic and non-enzymatic ingredients, and the like), and the enzyme(s) should be present in effective amounts. The medical cleaning compositions are commercially available.

### Dr. Weigert Neodisher Multizym (Dr. Weigert)

5-15% non-ionic surfactants, less than 5% anionic surfactants, enzymes, preservation agents: 2-Octyl-2H-isothiazol-3-one and methylisothiazolinone.

### Aniosyme Synergy 5 (Anios Laboratoires)

2.5-10% N,N-dimethyldecylamine N-oxide, less than 2.5% sodium methane sulphonate, less than 2.5% dicethyldioctylammonium chloride and less than 2.5% of a 3:1 mixture of 5-chlor-2-methyl-2H-isothiazol-3-on and 2-methyl-2H-isothiazol-3-on.

### Metrex Metrizyme (Metrex)

10-30% propylene glycol, 10-20% non-ionic surfactant, 5-10% fragrance oil, 1.1-1% proteinase substilisin and a 30-60% of a mixture of water and non-harzadous ingredients.

### Steris Prolystica 2X Enzymatic Presoak and Cleaner (Steris)

1-5% alcohols, C9-11, ethoxylated surfactant,
1-5% glycerin, 1-5% citric acid, 1-2% N,N-dimethyloctadecylamine oxide and 0.1-1% substilisins (proteolytic enzyme).

### Enzym E Assays

### Assay I: Testing of DNase Activity

DNase activity was determined on DNase Test Agar with Methyl Green (BD, Franklin Lakes, NJ, USA), which was prepared according to the manual from supplier. Briefly, 21 g of agar was dissolved in 500 ml water and then autoclaved for 15 min at 121° C. Autoclaved agar was temperated to 48° C. in water bath, and 20 ml of agar was poured into petridishes with and allowed to solidify by incubation o/n (over night) at room temperature. On solidified agar plates, 5 µl of enzyme solutions are added, and DNase activity are observed as colorless zones around the spotted enzyme solutions.

### EXAMPLES

### Example 1

### Removal Effect of DNase and Other Enzymes in Model Detergent on Pseudomonas Aeruginosa Dead Biofilm

Two different biofilms were used in the example, one produced by Pseudomonas aeruginosa PA14 (DSM19882) and one produced by P. aeruginosa PA01 (DSM22644). The bacteria were re-streaked on tryptic soy agar plates and incubated for 3 days at 30° C. After 3 days of incubation, tryptic soy broth (TSB) was inoculated with one colony of P. aeruginosa PA14 (DSM19882) or one colony of P. aeruginosa PA01 (DSM22644). The inoculated TSB tubes were all incubated overnight at 30° C. The overnight cultures were diluted in TSB to a specific optical density (OD). The diluted overnight culture was added to each well in Thermo Scientific™ Nunc™ MicroWell™ 96-Well Microplates (sterile, non-treated). The plates were incubated at 30° C. for 4 hours.

After 4 hours of incubation the microtiter plates (MTP) containing biofilm were removed from the incubator and emptied for media using Vacusafe™ Vacuum Aspiration System (INTEGRA Biosciences). Each well was rinsed twice with 0.9% NaCl solution. After rinse, 1000 ppm peracetic acid (PAA) (pH 3) was added to each well for complete kill of the bacteria. The MTPs were incubated with PAA for 10 minutes at 30° C. After PAA treatment, each well was rinsed twice with 0.9% NaCl solution.

The complete kill of microorganism was verified by PAA treatment of a biofilm grown under the same conditions as described above. After treatment with 1000 ppm PAA for 10 minutes and rinsing twice with 0.9% NaCl in each well, fresh TSB was added to each well. The MTP was placed at 30° C. for 4 days, whereafter each well was checked for regrowth by measuring the OD at 595 nm. No regrowth was observed in any of the wells.

A model detergent liquor was prepared by dissolving 5 g/l model detergent containing 5% MPG (mono propylene glycol), 5% Pluronic PE 4300 (PO/EO block polymer; 70%/30%, approx. 1750 g/mol), 2% Plurafac LF 305 (fatty alcohol alkoxylate; C6-10 + EO/PO), 1% MGDA (methyl glycine diacetic acid, 1% TEA (triethanolamine), pH-adjusted to 8.7 with phosphoric acid and water to 100% (all percentages are w/w (weight volume)) in water with hardness 5°dH.

Treatment solutions consisting of model detergent liquor ± enzymes were prepared. The treatment solutions (model detergent liquor ± enzymes) were added to each well in the MTPs. Each enzyme was applied at a final concentration of 5 µg/mL. Each treatment was applied to eight wells.

After addition of model detergent liquor +/- enzyme, the MTPs were incubated statically for 60 minutes at 30° C. After 60 minutes of incubation, the treatment liquor was removed using the vacuum system. Each well was rinsed twice with 0.9% NaCl solution, and 0.095% crystal violet solution was added to each well. The MTPs were incubated for 15 minutes at room temperature whereafter the crystal violet solution was removed using the vacuum system. Each well was rinsed twice with 0.9% NaCl solution and 30% acetic acid was added to each well. The MTPs were incubated for 10 minutes at room temperature, whereafter the absorbance at 595 nm was measured using a spectrophotometer (SpectraMax M3, Molecular Devices). The MTPs were shaked for 10 seconds before absorbance measurements were performed.

The enzymes tested in Example 1 are listed in Table 1 below.

TABLE 1

Protease
Mannanase
DNase SEQ ID NO: 2

The % remaining dead biofilm after enzymatic treatment was calculated as ABS_{595(biofilm} _{treated} _{with} _{model} _{detergent+enzyme)}/ABS_{595(biofilm} _{treated} _{with} _{model} _{detergent)} × 100%.

The results are displayed in Table 2 and Figure.

TABLE 2

Percent remaining dead biofilm after treatment with DNase, protease and/or mannanase in 0.5% model detergent wash liquor

| | P. aeruginosa PA14 (DSM 19882) | P. aeruginosa PA01 (DSM22644) |
|---|---|---|
| 0.5% Model detergent | 100 | 100 |
| 0.5% Model detergent + 5 µg/mL protease | 74 | 108 |
| 0.5% Model detergent + 5 µg/mL Mannanase | 87 | 100 |
| 0.5% Model detergent + 5 µg/mL DNase SEQ ID NO:2 | 11 | 9 |
| 0.5% Model detergent + 5 µg/mLprotease + 5 µg/mL Mannanase | 72 | 102 |
| 0.5% Model detergent + 5 µg/mL protease + 5 µg/mL + DNase SEQ ID NO: 2 | 8 | 7 |
| 0.5% Model detergent + 5 µg/mL Mannanase + 5 µg/mL DNase SEQ ID NO: 2 | 11 | 12 |
| 0.5% Model detergent + 5 µg/mL protease + 5 µg/mL Mannanase + 5 µg/mL DNase SEQ ID NO: 2 | 8 | 5 |

### Example 2

### Dosage Response Removal Effect of DNases in Model Detergent On Pseudomonas Aeruginosa Dead Biofilm

4-hour old and 24-hour old P. aeruginosa PA14 (DSM19882) and P. aeruginosa PA01 (DSM22644) dead biofilms were produced as described in Example 1, except that both aeruginosa strains were grown for 4 hours and 24 hours in MTPs before kill using 1000 ppm PAA.

After kill using PAA and rinsing with 0.9% NaCl, treatment solutions (model detergent liquor ± enzymes) were added to each well. The DNases were applied individually in model detergent liquor (as described in Example 1) in dilutions from 0 - 5 µg/mL. Each treatment was applied to eight wells. After addition of model detergent +/- enzyme, the microtiter plates were incubated statically for 60 minutes at 30° C. After 60 minutes of incubation, the remaining amount of dead biofilm was quantified, as described in Example 1, by staining with crystal violet and resolubilizing in 30% acetic acid. Finally, the absorbance was measured at 595 nm.

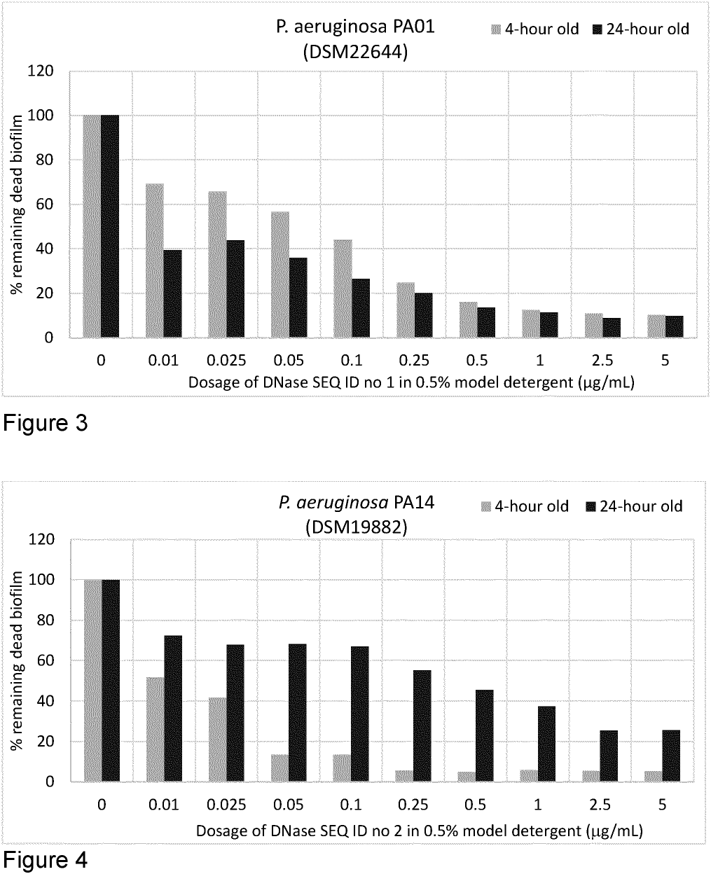

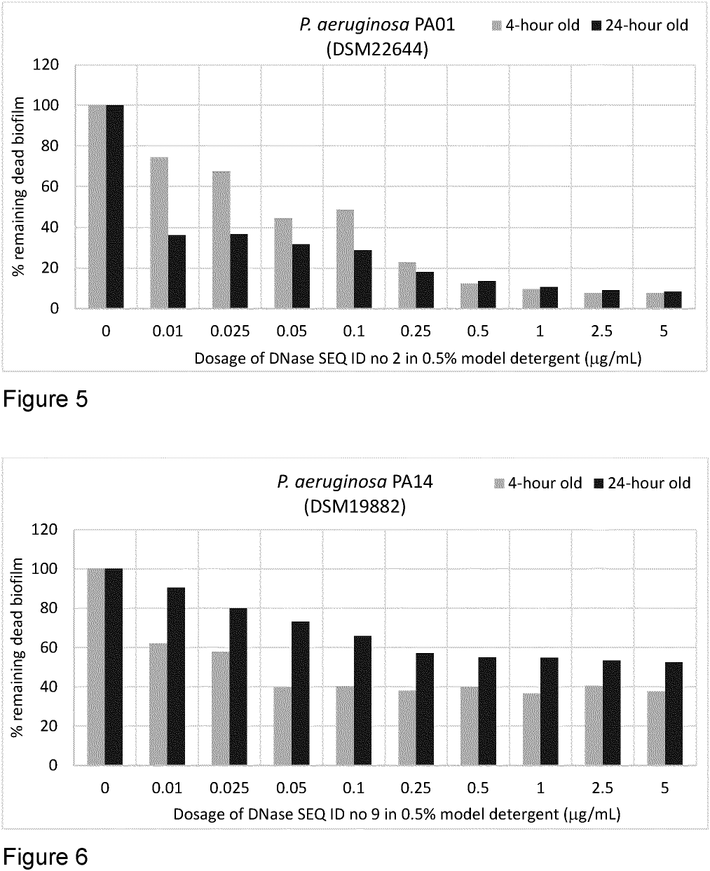

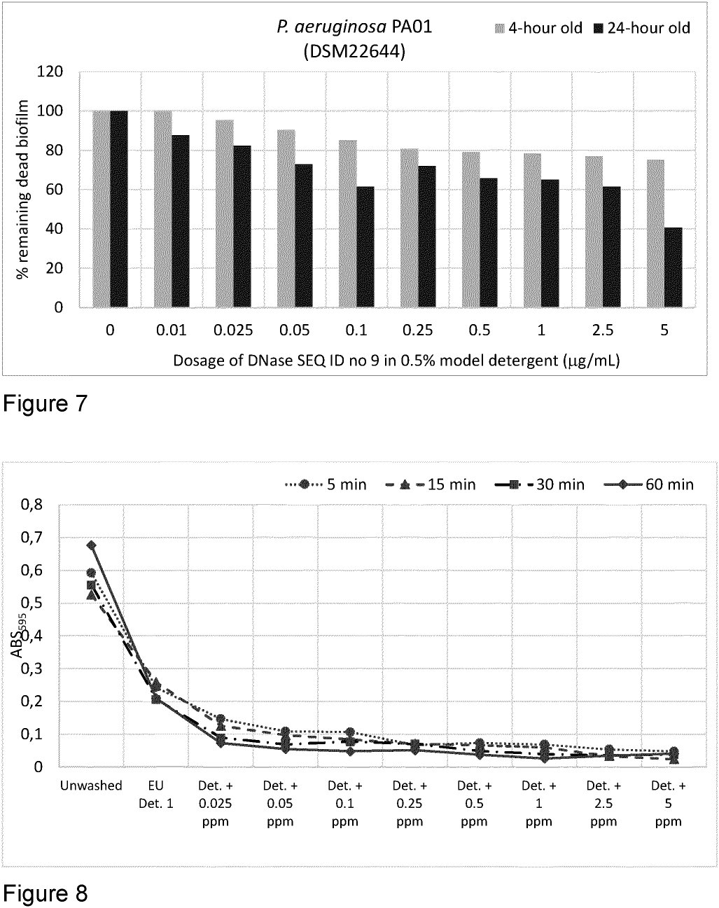

The percent remaining dead biofilm after enzymatic treatment was calculated as ABS_{595(biofilm} _{treated} _{with} _{model} _{detergent+enzyme)}/ABS_{595(biofilm} _{treated} _{with} _{model} _{detergent)} × 100%. The results are displayed in Tables 3-5 and in FIGS. 2-7.

TABLE 3

Percent remaining dead biofilm after treatment with increasing dosages of DNase SEQ ID NO: 1 in 0.5% model detergent

| | P. aeruginosa PA14 (DSM 19882) | | P. aeruginosa PA01 (DSM22644) | |
|---|---|---|---|---|
| Dosage of DNase SEQ ID NO: 1 (mg/L) | 4-hour old biofilm (dead) | 24-hour old biofilm (dead) | 4-hour old biofilm (dead) | 24-hour old biofilm (dead) |
| 0 | 100 | 100 | 100 | 100 |
| 0.01 | 39 | 45 | 69 | 39 |
| 0.025 | 28 | 54 | 66 | 44 |
| 0.05 | 24 | 43 | 57 | 36 |
| 0.1 | 20 | 37 | 44 | 26 |
| 0.25 | 14 | 28 | 25 | 20 |
| 0.5 | 10 | 25 | 16 | 14 |
| 1 | 8 | 20 | 12 | 11 |
| 2.5 | 7 | 18 | 11 | 9 |
| 5 | 6 | 18 | 10 | 10 |

TABLE 4

Percent remaining dead biofilm after treatment with increasing dosages of DNase SEQ ID NO: 2 in 0.5% model detergent

| | P. aeruginosa PA14 (DSM 19882) | | P. aeruginosa PA01 (DSM22644) | |
|---|---|---|---|---|
| Dosage of DNase SEQ ID NO: 2 (mg/L) | 4-hour old biofilm (dead) | 24-hour old biofilm (dead) | 4-hour old biofilm (dead) | 24-hour old biofilm (dead) |
| 0 | 100 | 100 | 100 | 100 |
| 0.01 | 52 | 72 | 74 | 36 |
| 0.025 | 42 | 68 | 68 | 37 |
| 0.05 | 14 | 68 | 45 | 32 |
| 0.1 | 14 | 67 | 49 | 29 |
| 0.25 | 6 | 55 | 23 | 18 |
| 0.5 | 5 | 45 | 12 | 14 |
| 1 | 6 | 37 | 10 | 11 |
| 2.5 | 6 | 25 | 8 | 9 |
| 5 | 5 | 26 | 8 | 8 |

TABLE 5

Percent remaining dead biofilm after treatment with increasing dosages of DNase SEQ ID NO: 9 in 0.5% model detergent

| | P. aeruginosa PA14 (DSM 19882) | | P. aeruginosa PA01 (DSM22644) | |
|---|---|---|---|---|
| Dosage of DNase SEQ ID NO: 9 (mg/L) | 4-hour old biofilm (dead) | 24-hour old biofilm (dead) | 4-hour old biofilm (dead) | 24-hour old biofilm (dead) |
| 0 | 100 | 100 | 100 | 100 |
| 0.01 | 62 | 91 | 100 | 88 |
| 0.025 | 58 | 80 | 95 | 82 |
| 0.05 | 40 | 73 | 90 | 73 |
| 0.1 | 40 | 66 | 85 | 61 |
| 0.25 | 38 | 57 | 81 | 72 |
| 0.5 | 40 | 55 | 79 | 66 |
| 1 | 37 | 55 | 78 | 65 |
| 2.5 | 41 | 53 | 77 | 61 |
| 5 | 38 | 53 | 75 | 41 |

### Example 3

### Effect of Time and Dosage Response of DNase in Commercial Detergents On Dead Pseudomonas Aeruginosa Biofilm

In this example, the dead biofilm removal effect of DNase was tested in four different commercially available endoscopic reprocessing detergents, two detergents from EU and two detergents from US. Dead biofilm from P. aeruginosa PA14 was produced for 4 hours as described in Example 1. DNase was applied in each detergent to the dead biofilm in dosages from 0-5 for 5 minutes, 15 minutes, 30 minutes and 60 minutes. The detergents were applied in the middle of the recommended dosage range. Unwashed control wells were included in the examples, where 200 µl 0.9% NaCl solution was added to the unwashed wells during the treatment time. The remaining dead biofilm was quantified as described in Example 1 by staining with crystal violet and resolubilizing in 30% acetic acid. The absorbance was measured at 595 nm, where a high amount of remaining biofilm results in a high absorbance value.

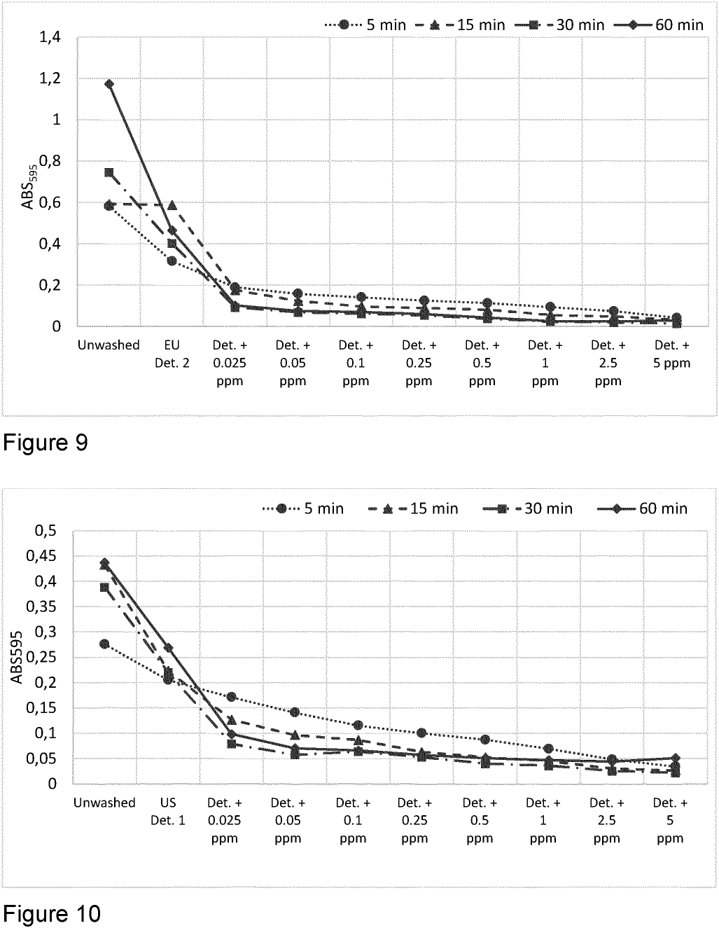

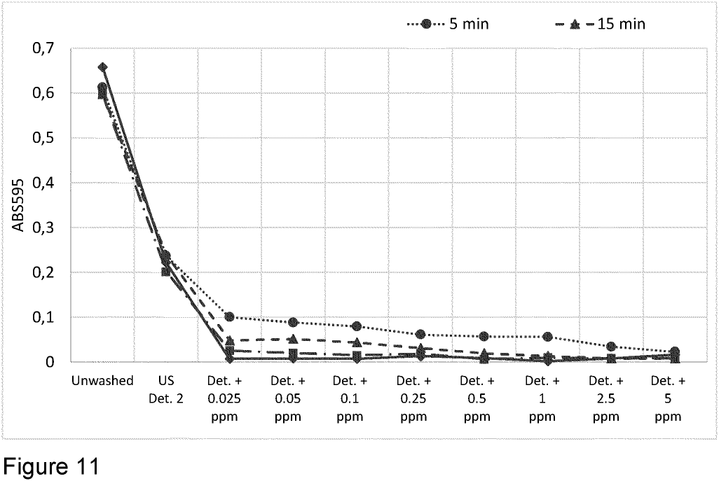

The results are displayed in Tables 6-9 and in FIGS. 8-11 as absorbance values.

TABLE 6

ABS₅₉₅ₙₘ of crystal violet stained remaining dead biofilm after treatment with increasing dosage of DNase SEQ ID NO: 2 in EU detergent 1, Dr. Weigert Neodisher Multizym, for 5, 15, 30 and 60 minutes
Treatment time

| Treatment | 5 min | 15 min | 30 min | 60 min |
|---|---|---|---|---|
| Unwashed | 0.592 | 0.526 | 0.556 | 0.676 |
| EU detergent 1 | 0.243 | 0.259 | 0.207 | 0.209 |
| EU detergent 1 + 0.025 ppm DNase SEQ ID NO: 2 | 0.146 | 0.125 | 0.089 | 0.072 |
| EU detergent 1 + 0.05 ppm DNase SEQ ID NO: 2 | 0.108 | 0.097 | 0.069 | 0.055 |
| EU detergent 1 + 0.1 ppm DNase SEQ ID NO: 2 | 0.107 | 0.084 | 0.078 | 0.048 |
| EU detergent 1 + 0.25 ppm DNase SEQ ID NO: 2 | 0.067 | 0.071 | 0.071 | 0.051 |
| EU detergent 1 + 0.5 ppm DNase SEQ ID NO: 2 | 0.073 | 0.067 | 0.049 | 0.037 |
| EU detergent 1 + 1 ppm DNase SEQ ID NO: 2 | 0.068 | 0.060 | 0.040 | 0.026 |
| EU detergent 1 + 2.5 ppm DNase SEQ ID NO: 2 | 0.053 | 0.033 | 0.036 | 0.034 |
| EU detergent 1 + 5 ppm DNase SEQ ID NO: 2 | 0.048 | 0.024 | 0.039 | 0.041 |

TABLE 7

ABS₅₉₅ₙₘ of crystal violet stained remaining dead biofilm after treatment with increasing dosage of DNase SEQ ID NO: 2 in EU detergent 2, Aniosyme Synergy 5, for 5, 15, 30 and 60 minutes
Treatment time

| Treatment | 5 min | 15 min | 30 min | 60 min |
|---|---|---|---|---|
| Unwashed | 0.582 | 0.593 | 0.746 | 1.173 |
| EU detergent 2 | 0.316 | 0.587 | 0.402 | 0.465 |
| EU detergent 2 + 0.025 ppm DNase SEQ ID NO: 2 | 0.189 | 0.175 | 0.093 | 0.101 |
| EU detergent 2 + 0.05 ppm DNase SEQ ID NO: 2 | 0.158 | 0.123 | 0.069 | 0.075 |
| EU detergent 2 + 0.1 ppm DNase SEQ ID NO: 2 | 0.141 | 0.097 | 0.062 | 0.069 |
| EU detergent 2 + 0.25 ppm DNase SEQ ID NO: 2 | 0.125 | 0.090 | 0.055 | 0.060 |
| EU detergent 2 + 0.5 ppm DNase SEQ ID NO: 2 | 0.113 | 0.080 | 0.038 | 0.042 |
| EU detergent 2 + 1 ppm DNase SEQ ID NO: 2 | 0.094 | 0.056 | 0.024 | 0.025 |
| EU detergent 2 + 2.5 ppm DNase SEQ ID NO: 2 | 0.074 | 0.049 | 0.019 | 0.024 |
| EU detergent 2 + 5 ppm DNase SEQ ID NO: 2 | 0.042 | 0.031 | 0.014 | 0.028 |

TABLE 8

ABS₅₉₅ₙₘ of crystal violet stained remaining dead biofilm after treatment with increasing dosage of DNase SEQ ID NO: 2 in US detergent 1, Steris Prolysitica 2X Enzymatic Presoak and Cleaner, for 5, 15, 30 and 60 minutes
Treatment time

| Treatment | 5 min | 15 min | 30 min | 60 min |
|---|---|---|---|---|
| Unwashed | 0.276 | 0.432 | 0.389 | 0.437 |
| US detergent 1 | 0.205 | 0.223 | 0.220 | 0.269 |
| US detergent 1 + 0.025 ppm DNase SEQ ID NO: 2 | 0.171 | 0.126 | 0.079 | 0.098 |
| US detergent 1 + 0.05 ppm DNase SEQ ID NO: 2 | 0.141 | 0.096 | 0.058 | 0.070 |
| US detergent 1 + 0.1 ppm DNase SEQ ID NO: 2 | 0.115 | 0.086 | 0.064 | 0.066 |
| US detergent 1 + 0.25 ppm DNase SEQ ID NO: 2 | 0.100 | 0.063 | 0.053 | 0.057 |
| US detergent 1 + 0.5 ppm DNase SEQ ID NO: 2 | 0.087 | 0.053 | 0.040 | 0.051 |
| US detergent 1 + 1 ppm DNase SEQ ID NO: 2 | 0.069 | 0.045 | 0.036 | 0.047 |
| US detergent 1 + 2.5 ppm DNase SEQ ID NO: 2 | 0.048 | 0.030 | 0.026 | 0.044 |
| US detergent 1 + 5 ppm DNase SEQ ID NO: 2 | 0.034 | 0.027 | 0.022 | 0.051 |

TABLE 9

ABS₅₉₅ₙₘ of crystal violet stained remaining dead biofilm after treatment with increasing dosage of DNase SEQ ID NO: 2 in US detergent 2, Metrex Metrizyme, for 5, 15, 30 and 60 minutes
Treatment time

| Treatment | 5 min | 15 min | 30 min | 60 min |
|---|---|---|---|---|
| Unwashed | 0.613 | 0.598 | 0.599 | 0.658 |
| US detergent 2 | 0.237 | 0.239 | 0.202 | 0.222 |
| US detergent 2 + 0.025 ppm DNase SEQ ID NO: 2 | 0.100 | 0.048 | 0.026 | 0.007 |
| US detergent 2 + 0.05 ppm DNase SEQ ID NO: 2 | 0.088 | 0.052 | 0.020 | 0.008 |
| US detergent 2 + 0.1 ppm DNase SEQ ID NO: 2 | 0.080 | 0.044 | 0.015 | 0.007 |
| US detergent 2 + 0.25 ppm DNase SEQ ID NO: 2 | 0.061 | 0.032 | 0.019 | 0.013 |
| US detergent 2 + 0.5 ppm DNase SEQ ID NO: 2 | 0.057 | 0.020 | 0.006 | 0.009 |
| US detergent 2 + 1 ppm DNase SEQ ID NO: 2 | 0.056 | 0.014 | 0.010 | 0.002 |
| US detergent 2 + 2.5 ppm DNase SEQ ID NO: 2 | 0.034 | 0.008 | 0.009 | 0.008 |
| US detergent 2 + 5 ppm DNase SEQ ID NO: 2 | 0.023 | 0.009 | 0.009 | 0.017 |

1. A method for cleaning a medical device, comprising the steps of:
a) providing a wash liquor having a pH in the range of 6.0-10.0, wherein the wash liquor comprises a polypeptide having DNase activity and optionally further cleaning components,
b) subjecting at least part of the medical device to the wash liquor of step a),
c) optionally, subjecting the medical device to rinsing,
d) optionally, subjecting the medical device to drying, and
e) optionally, subjecting the medical device to sterile packaging.

2. The method according to claim 1, wherein at least a part of the medical device comprises organic soil.

3. The method according to claim 2, wherein the wash liquor comprising a polypeptide having DNase activity releases and/or removes at least part of the organic soil present on the part of the medical device subjected to the wash liquor.

4. The method according to claim 1, wherein the medical device is disinfected before being subjected to the wash liquor.

5. The method according to claim 1, wherein at least part of the medical device is subjected to a liquid solution comprising peracetic acid having a pH of about 3, whereafter no live microorganisms are comprised in the organic soil.

6. The method according to claim 1, wherein the wash liquor comprising a polypeptide having DNase activity releases and/or removes at least 50% of the organic soil present on the part of the medical device subjected to the wash liquor as measured in Example 2, preferably at least 60% of the organic soil is released and/or removed, more preferably at least 70% of the organic soil is released and/or removed, even more preferably at least 80% of the organic soil is released and/or removed, most preferably at least 90% of the organic soil is released and/or removed, such as at least 95% of the organic soil is released and/or removed.

7. The method according to claim 1, wherein the polypeptide having DNase activity is of bacterial or fungal origin.

8. The method according to claim 1, wherein the polypeptide having DNase activity is selected from the group consisting of:
a) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 1;
b) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 2;
c) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 3;
d) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 4;
e) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 5;
f) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 6;
g) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 7;
h) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 8;
i) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 9, and
j) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 10.

9. A medical cleaning composition comprising a polypeptide having DNase activity, wherein the composition has a pH in the range of 6.0-10.0.

10. A medical cleaning composition according to claim 9, wherein the polypeptide is selected from the group consisting of:
a) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 1;
b) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 2;
c) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 3;
d) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 4;
e) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 5;
f) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 6;
g) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 7;
h) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 8;
i) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 9, and
j) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide shown in SEQ ID NO: 10.

11. The medical cleaning composition according to claim 9, wherein the composition further comprises one or more cleaning components, preferably selected from surfactants, builders, bleach components, polymers, dispersing agents and additional enzymes.

12. The medical cleaning composition according to claim 9, wherein the medical cleaning composition further comprises one or more enzymes selected from the group consisting of proteases, amylases, lipases, cutinases, cellulases, endoglucanases, xyloglucanases, pectinases, pectin lyases, xanthanases, peroxidases, haloperoxygenases, catalases, galactanase, mannanases, or any mixture thereof.

13. The medical cleaning composition according to claim 9, the composition comprising 5-25% of a non-ionic surfactant and 0-5% of an anionic surfactant.

14. (canceled)

15. A medical device coated with a composition comprising a polypeptide having DNase activity.

16. A method of releasing and/or removing organic soil from a medical device, the method comprising contacting a soiled medical device with a polypeptide having DNase activity.
